(19) United States
(12) Patent Application Publication
(10) Pub. No.: US 2011/0281765 A1
(43) Pub. Date: Nov. 17, 2011

(54) Plant polymorphic markers and uses thereof

(76) Inventors: David F. Bush, Chicago, IL (US); Steven D. Rounsley, Boylston, MA (US); Roger C. Wiegand, Wayland, MA (US)

(21) Appl. No.: 10/746,294

(22) Filed: Dec. 29, 2003

Related U.S. Application Data
(63) Continuation-in-part of application No. 09/692,412, filed on Oct. 20, 2000, now abandoned.
Application No. 09/692,412 is a continuation-in-part of application No. 09/534,859, filed on Mar. 29, 2000, now abandoned.
Application No. 09/534,859 is a continuation-in-part of application No. 09/803,736, filed on Mar. 12, 2001, now abandoned.

(51) Int. Cl. C40B 40/06; C07H 1/08; C40B 30/04

(52) U.S. Cl. 506/16; 506/9; 536/25.4

(57) ABSTRACT

The present invention is in the field of plant genetics. More specifically, the invention relates to nucleic acid markers associated with Arabidopsis thaliana ecotypes. The invention also relates to methods for detecting polymorphisms. The invention further relates to methods of using nucleic acid markers, for example, for genome mapping, gene identification, gene isolation and gene analysis.

### CROSS REFERENCE TO RELATED APPLICATION

This application is a continuation-in-part of application Ser. No. 09/692,412 filed Oct. 20, 2000, and a continuation in part of application Ser. No. 09/534,859, filed Mar. 29, 2000, and a continuation in part of application Ser. No. 09/803,736, filed Mar. 12, 2001, all of which are herein incorporated by reference in their entirety.

### INCORPORATION OF SEQUENCE LISTING

This application contains a sequence listing, which is contained on three identical CD-Rs: two copies of a sequence listing (Seq. listing: Copy 1 and Seq. listing: Copy 2) and a sequence listing Computer Readable Form (CRF), all of which are herein incorporated by reference. All three CD-Rs each contain one file called “marker patent.txt” which is 13,814,237 bytes in size (measured in MS-DOS) and was created on Dec. 23, 2003.

### INCORPORATION OF TABLE A

Two copies each of Table A on CD-Rs, each containing two files: (1) Table 1A.doc which is 654,848 bytes (measured in MS-DOS) created on Dec. 23, 2003; and (2) Table 1B.doc which is 751,104 bytes (measured in MS-DOS) created on Dec. 23, 2003, are herein incorporated by reference.

LENGTHY TABLES
The patent application contains a lengthy table section. A copy of the table is available in electronic form from the USPTO web site (http://seqdata.uspto.gov/?pageRequest=docDetail&DocID=US20110281765A1). An electronic copy of the table will also be available from the USPTO upon request and payment of the fee set forth in 37 CFR 1.19(b)(3).

### FIELD OF THE INVENTION

The present invention is in the field of plant genetics. More specifically, the invention relates to nucleic acid markers associated with Arabidopsis thaliana ecotypes. The invention also relates to methods for detecting polymorphisms and using polymorphic markers, for genotyping applications, e.g. identifying and isolating regions of DNA associated with phenotypic traits.

### BACKGROUND OF THE INVENTION

I. Arabidopsis thaliana

The identification in Arabidopsis thaliana of polymorphic markers is important in the development of nutritionally enhanced or agriculturally enhanced crops. Such polymorphic markers are useful in, for example, genetic mapping or linkage analysis, marker assisted breeding, physical genome mapping, transgenic crop production, crop monitoring diagnostics, and gene identification and isolation.

Arabidopsis thaliana is widely used as a model organism for basic and applied research in the biology of flowering plants. Arabidopsis thaliana is a model system for plant genomic research in part due to its small and characterized genome, which is estimated to be comprised of approximately 20,000 to 25,000 genes. The genome is estimated to have a haploid content of around 100 Mb, present on five chromosomes. Reported partial sequence analysis has provided information on genome features such as gene density and gene structure (Settles and Byrne, Genome Research 8:83-85 (1998), the entirety of which is herein incorporated by reference). Based on reports from the European Union Sequencing Consortium, the average gene density is one gene every approximately 4.8 kb.

Other important characteristics that make Arabidopsis thaliana a useful test system include its rapid life-cycle, small size, which allows for controlled growth in restricted space, its prolific seed production, the availability of characterized and uncharacterized mutants and the existence of a reliable transformation system.

Molecular genetics is often used in the analysis of plant genes and is particularly useful in the analysis of complex biological processes such as developmental regulation. In one approach the use of mutant plants, e.g. Arabidopsis thaliana mutants, in molecular genetic research requires the location of the mutation. Molecular markers are a useful way to locate such mutations.

Identification of target loci and the isolation of associated genes using molecular markers has been reported (Liu et al., Proc. Natl. Acad. Sci. USA, 96:6535-6540 (1999); Muramoto et al., The Plant Cell, 11:335-347 (1999); Bowman and Smyth, Development, 126:2387-2396 (1999); Michaels and Amasino, The Plant Cell, 11:949-956 (1999); Ha et al., The Plant Cell, 11:1153-1163 (1999); Walker et al., The Plant Cell, 11:1337-1349 (1999); Sedbrook et al., Proc. Natl. Acad. Sci. USA, 96:1140-1145 (1999); Kiyosue et al., Proc. Natl. Acad. Sci. USA, 96:4186-4191 (1999); and Davis et al., Proc. Natl. Acad. Sci. USA, 96:6541-6546 (1999), all of which are herein incorporated by reference in their entirety). The use of markers to isolate a genomic region of interest is often referred to as map based cloning, chromosome walking or positional cloning. Many of the Arabidopsis thaliana markers that have been used in map based cloning are anchored to genetic maps such as the Lister & Dean map (See e.g. genome-www3.stanford.edu/cgi-bin/AtDB/RIintromap).

Physical or partial physical maps of the Arabidopsis thaliana genome have also been reported (See e.g. genome-www3.stanford.edu/atdb_welcome.html). A physical map of Arabidopsis thaliana, Columbia based on a collection of bacterial artificial chromosomes (BACs) is available (Marra et al., Nat. Genet., 22(3):265-270 (1999); Mozo et al., Nat. Genet., 22(e):271-275 (1999), both of which are herein incorporated by reference in their entirety). An overlapping series of BACs representing the Arabidopsis thaliana, Columbia genome is available from AIMS, Arabidopsis Biological Resource Center, 309 B&Z Building, 1735 Neil Avenue, Columbus, Ohio 43210, USA.

Cho et al. reported a low density biallelic polymorphic map based on a comparison of Arabidopsis thaliana, Columbia and Arabidopsis thaliana, Landsberg erecta ecotypes by screening approximately 0.5% of the genome for such polymorphisms (Cho et al., Nature Genetics 23:203-207 (1999), the entirety of which is herein incorporated by reference). In this survey 487 single nucleotide polymorphisms (SNPs) were reported. Cho et al. also reported the use of oligonucleotide arrays to detect Arabidopsis thaliana SNPs.

The present invention provides polymorphic nucleic acid markers whose physical location is known within the Arabidopsis thaliana genome. Moreover, the physical location of such markers is further known within a particular BAC and the position of that BAC relative to other BACs in the genome is also known.

Successful isolation of a region of Arabidopsis thaliana DNA associated with a trait of interest requires a nucleic acid marker to be sufficiently close to the trait. As the present invention provides a collection of nucleic acid markers in the Arabidopsis thaliana genome which allows for the efficient isolation of regions of Arabidopsis thaliana DNA associated with traits of interest. Moreover, the association of a collection of nucleic acid markers with a trait of interest may be simultaneously investigated.

### SUMMARY OF THE INVENTION

The present invention provides a collection of nucleic acid molecules capable of detecting a set of polymorphisms as shown in Table A.

The present invention also includes and provides a method of isolating a region of genomic DNA associated with a phenotype of interest comprising: (A) identifying an Arabidopsis plant of a first ecotype with a phenotype of interest; (B) crossing said Arabidopsis plant with an Arabidopsis plant of a second ecotype lacking said phenotype; (C) propagating and self pollinating seeds from said cross; (D) selecting progeny of self pollinated seeds with said phenotype; (E) screening progeny of self pollinated seeds with said phenotype with a collection of nucleic acid molecules, said collection of nucleic acid molecules capable of detecting a set of polymorphisms where the polymorphisms are distributed throughout the genome of said self pollinated seeds with said phenotype at an average density of more than one polymorphism per about 100 kb, wherein at least one of the polymorphisms is selected from Table A; (F) calculating the linkage of each of said polymorphisms to said phenotype; and (G) isolating said region of genomic DNA associated with said phenotype based on its linkage to one or more of said nucleic acid molecules.

The present invention also provides a method of identifying a region of genomic DNA associated with a phenotypic trait of interest comprising: (A) screening a mapping populatini of Arabidopsis plants to determine the linkage of the phenotypic trait with a coolection of nucleic acid molecules, wherein the nucleic acid molecules are capable of detecting a set of polymorphisms, where the polymorphisms are distributed throughout the genome of the mapping population of Arabidopsis plants at an average density of more than one polymorphism per about 100 kb, wherein at least on eof the polymorphisms is selected from Table A; (B) calculating the linkage of each of the polymorphisms to the phenotypic trait; and (C) identifying the genomic DNA region associated with said phenotypic trait based on its linkage to one or more of said nucleic acid molecules.

The present invention also provides a method of identifying a nucleic acid molecule associated with a phenotypic trait of interest comprising: (A) screening a mapping population of Arabidopsis plants to determine the linkage of said phenotypic trait with a collection of polymorphisms, wherein said polymorphisms are distributed throughout the genome of said mapping population of Arabidopsis plants at an average density of more than one polymorphism per about 100 kb, wherein at least one of the polymorphisms is selected from Table A; (B) calculating the linkage of each of said polymorphisms to said phenotypic trait; and (C) isolating said nucleic acid molecule associated with said phenotypic trait based on its linkage to one or more of said polymorphisms.

The present invention also provides a method of isolating a nucleic acid molecule associated with a phenotypic trait comprising: (A) screening a mapping population of Arabidopsis plants to determine the linkage of said phenotypic trait with a collection of polymorphisms, wherein at least one polymorphism is selected form Table A; and (B) isolating said nucleic acid molecule associated with said phenotypic trait based on its linkage to one or more of said polymorphisms.

The present invention further provides a collection of non-identical nucleic acid molecules capable of detecting polymorphisms present in an Arabidopsis mapping population, wherein said collection of non-identical nucleic acid molecules is capable of detecting at least 25 polymorphisms selected from the group consisting of Table A. Such a collection may also be recorded on a computer readable medium having recorded thereon at least 100 of the polymorphisms set forth in Table A. Such a collection may also be deposited on a substrate.

The present invention also provides a method of introgressing a trait of interest into a plant comprising using a nucleic acid marker for marker assisted selection of said plant, said nucleic acid marker capable of detecting a polymorphism selected from Table A, and introgressing said trait into said plant.

The present invention further provides a method for identifying transposons in the DNA of an organism comprising identifying INDELs in said DNA and comparing the sequence of said INDELs to the sequence of one or more known transposons.

The present invention also provides a method of identifying a nucleic acid molecule associated with a phenotypic trait comprising: (A) screening a mapping population of Arabidopsis plants to determine the linkage of said phenotypic trait with a collection of polymorphisms, where the polymorphisms are distributed throughout the genome of said mapping population of Arabidopsis plants at an average density of more than one polymorphism per about 100 kb; and (B) identifying said nucleic acid molecule associated with said phenotypic trait based on its linkage to one or more of said polymorphisms.

Also provided is a method of identifying a nucleic acid molecule associated with a phenotypic trait of interest comprising: (A) screening a mapping population of Arabidopsis plants to determine the linkage of said phenotypic trait with a collection of polymorphisms, wherein said polymorphisms are distributed throughout the genome of said mapping population of Arabidopsis plants wherein at least one of the polymorphisms is selected from Table A; (B) calculating the linkage of each of said polymorphisms to said phenotypic trait; and, (C) isolating said nucleic acid molecule associated with said phenotypic trait based on its linkage to one or more of said polymorphisms, and wherein said collection of polymorphisms is designed from features of polymorphisms listed in Table A. Such a method may also be recorded on computer readable medium.

The present invention also provides a computer readable medium having recorded thereon polymorphisms selected from the group consisting of Single Nucleotide Polymorphisms 466799, 471736, 459697, 459698, 459699, 459700, 459701, 445015, 459702, 459703, 445016, 445017, 461062, 447523, 461063, 447524, 447525, 461064, 461065, 447526, 461066, 450823, 450824, 450825, 450826, 428620, 450827, 462117, 450828, 462118, 428621, 428622, 428623, 450829, 462119, 428624, 428625, 428626, 450830, 450831, 450832, 450833, 448562, 424491, 424492, 424493, 424494, 424495, 424496, 424497, 448563, 448564, 424498, 424499, 448565, 461975, 424500, 424501, 424502, 424503, 448566, 448567, 448568, 448569, 448570, 424504, 424505, 424506, 424507, 424508, 424509, 424510, 424511, 448571, 424512, 424513, 424514, 424515, 448572, 426007, 426008, 426009, 426010, 449396, 462027, 449397, 449398, 426011, 426012, 449399, 449400, 462028, 449401, 462029, 449402, 426013, 426014, 426015, 426016, and 426017.

Also provided are a collection of non-identical nucleic acid molecules capable of detecting polymorphisms in an Arabidopsis thaliana mapping population, wherein said collection of non-identical nucleic acid molecules is capable of detecting polymorphisms selected from the group consisting of Single Nucleotide Polymorphisms 466799, 471736, 459697, 459698, 459699, 459700, 459701, 445015, 459702, 459703, 445016, 445017, 461062, 447523, 461063, 447524, 447525, 461064, 461065, 447526, 461066, 450823, 450824, 450825, and 450826.

The present invention also provides a computer readable medium having recorded thereon Single Nucleotide Polymorphisms 466799 or 471736.

The present invention further provides a computer readable medium having recorded thereon a set of polymorphisms wherein said polymorphisms are distributed throughout the genome of Arabidopsis at an average density of more than one polymorphism per about 100 kb.

### DETAILED DESCRIPTION OF THE INVENTION

The genomes of animals and plants naturally undergo spontaneous mutation in the course of their continuing evolution (Gusella, Ann. Rev. Biochem. 55:831-854 (1986), the entirety of which is herein incorporated by reference). A “polymorphism” is a variation or difference in the sequence of a genetic region that arises in some of the members of a species. Variant sequences can be defined with reference to an arbitrary or non-arbitrary standard sequence for the species. A polymorphism is thus said to be “allelic,” in that, due to the existence of the polymorphism, some members of a species may have the “standard” sequence (i.e. the standard “allele”) whereas other members may have a variant sequence (i.e., a variant “allele”). Thus, as used herein, an allele is one of two or more alternative versions of a gene or other genetic region at a particular location on a chromosome. In the simplest case, only one variant sequence may exist, and the polymorphism is thus said to be bi-allelic. In other cases, the species' population may contain multiple alleles, and the polymorphism is termed tri-allelic, etc.

A single gene or genetic region may have multiple different unrelated polymorphisms. For example, it may have a one bi-allelic polymorphism at one site, another bi-allelic polymorphism at another site and a multi-allelic polymorphism at another site. When all the sequences for a group of alleles at a chromosomal locus in a plant are the same, the alleles are said to be “homozygous” at that locus. When the sequence of any allele at a particular locus in a plant is different, the population of alleles is said to be “heterozygous” at that locus.

Phenotypic traits can vary due to environmental and/or genetic factors. For example, polymorphisms at a particular chromosomal locus can affect the phenotypic trait associated with that locus.

As used herein, a phenotypic trait of interest may be any trait exhibited by a plant, whether naturally occurring or otherwise, that is capable of being inherited. Moreover, the phenotypic trait of interest may, for example, be transient, permanent or only present when the plant or part thereof is subjected to environmental stimuli or challenge. A phenotypic trait of interest may be a desired trait. In other cases the phenotypic trait of interest may be an undesired trait. Furthermore, phenotypic traits are not limited to visible traits. While the phenotypic trait may be any trait, preferred traits of interest are those that have agricultural significance. Examples of agricultural traits include those that affect a component of yield, those that provide disease or chemical resistance, and those that affect developmental traits such as pollen or ovule production, etc., and those that affect composition of plants or plant parts, including seed proteins or oils, starch or sugar composition, nutrient content and the like.

Many phenotypic traits are the result of multiple genes or genetic factors, for example, a phenotypic trait that is the result of a quantitative trait allele. An allele of a quantitative trait locus (QTL) can, of course, comprise multiple genes or other genetic factors even within a contiguous genomic region or linkage group. As used herein, an allele of a quantitative trait locus can therefore encompass more than one gene or other genetic factor where each individual gene or genetic component is also capable of exhibiting allelic variation and where each gene or genetic factor also has a phenotypic affect on the quantitative trait in question.

As used herein, a “marker” is an indicator for the presence of at least one polymorphism. A marker is preferably a nucleic acid molecule. It is understood that a marker can, for example, be an oligonucleotide probe or primer.

A “nucleic acid marker” as used herein means a nucleic acid molecule that is capable of being a marker for detecting a polymorphism.

The term “oligonucleotide” as used herein refers to short nucleic acid molecules useful, e.g. for hybridizing probes, nucleotide array elements or amplification primers. Oligonucleotide molecules are comprised of two or more nucleotides, i.e. deoxyribonucleotides or ribonucleotides, preferably more than five and up to 30 or more. The exact size will depend on many factors, which in turn depend on the ultimate function or use of the oligonucleotide. Oligonucleotides can comprise ligated natural nucleic acid molecules or synthesized nucleic acid molecules and comprise between 10 to 150 nucleotides or between about 12 and about 100 nucleotides which have a nucleotide sequence which can hybridiaze to a strand of polymorphic DNA, e.g. to permit detection of a polymorphism. Such oligonucleotides may be nucleic acid elements for use on solid arrays (e.g. synthesized or spotted). In preferred aspects of the invention such oligonucleotides can comprise as few as 12 hybridizing nucleotides, e.g. for assays where the oligonucleotide also comprises a detectable label. In other preferred aspects of the invention the oligonucleotide can comprise as few as about 15 hybridizing nucleotides, e.g. for single base extension assays.

Such oligonucleotides may also be primers for use in polymerase chain reaction (PCR) or other reactions. The term “primer” as used herein refers to a nucleic acid molecule, preferably an oligonucleotide whether derived from a naturally occurring molecule such as one isolated from a restriction digest or one produced synthetically, which is capable of acting as a point of initiation of synthesis when placed under conditions in which synthesis of a primer extension product which is complementary to a nucleic acid strand is induced, i.e., in the presence of nucleotides and an agent for polymerization such as DNA polymerase and at a suitable temperature and pH. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the agent for polymerization. The exact lengths of the primers will depend on many factors, including temperature and source of primer. For example, depending on the complexity of the target sequence, the oligonucleotide primer typically contains at least 15, more preferably 18 nucleotides, which are identical or complementary to the template and optionally a tail of variable length which need not match the template. The length of the tail should not be so long that it interferes with the recognition of the template. Short primer molecules generally require cooler temperatures to form sufficiently stable hybrid complexes with the template.

The primers herein are selected to be “substantially” complementary to the different strands of each specific sequence to be amplified. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to be amplified to hybridize therewith and thereby form a template for synthesis of the extension product of the other primer. Computer generated searches using programs such as Primer3 (www-genome.wi.mit.edu/cgi-bin/primer/primer3.cgi), STSPipeline (www-genome.wi.mit.edu/cgi-bin/www-STS_Pipeline), or GeneUp (Pesole et al., BioTechniques 25:112-123 (1998), the entirety of which is herein incorporated by reference), for example, can be used to identify potential PCR primers. Exemplary primers include primers that are 18 to 50 bases long, where at least between 18 to 25 bases are identical or complementary to at least 18 to 25 bases of a segment of the template sequence.

This invention also contemplates and provides primer pairs for amplification of nucleic acid molecules in order to detect polymorphisms. As used herein “primer pair” means a set of two oligonucleotide primers based on two separated sequence segments of a target nucleic acid sequence. One primer of the pair is a “forward primer” or “5′ primer” having a sequence which is identical to the more 5′ of the separated sequence segments (+ strand). The other primer of the pair is a “reverse primer” or “3′ primer” having a sequence which is the reverse complement of the more 3′ of the separated sequence segments (+ strand). A primer pair allows for amplification of the nucleic acid sequence between and including the separated sequence segments. Optionally, each primer pair can comprise additional sequences, e.g. universal primer sequences or restriction endonuclease sites, at the 5′ end of each primer, e.g. to facilitate cloning, DNA sequencing, or reamplification of the target nucleic acid sequence.

As used herein, a “mapping population” is a collection of plants capable of being used with markers to map the genetic position of traits.

As used herein, a “polymorphic marker” is a marker capable of detecting one or more polymorphisms.

The present invention provides nucleic acid molecules which are markers, i.e. capable of detecting polymorphisms that are distributed throughout the genome of a mapping population.

As used herein, a “characterized polymorphism” is a polymorphism whose physical position on a genome is known. In a preferred embodiment, the physical position of a characterized polymorphism on an isolated nucleic acid molecule, such as a bacterial artificial chromosome comprising Arabidopsis thaliana genomic DNA, is known. Thus the present invention also provides nucleic acid molecules capable of detecting characterized polymorphisms throughout a genome.

In a further preferred embodiment, a characterized polymorphism is any polymorphism where the nucleic acid sequences of at least two of the polymorphisms present in an Arabidopsis mapping population are known (sequenced characterized polymorphism). In a particularly preferred embodiment, a characterized polymorphism is a polymorphism from Table A. In another particularly preferred embodiment, a characterized polymorphism from Table A is part of a collection of polymorphisms, where preferably over 25%, more preferably over 50% and even more preferably over 75% of the polymorphisms are selected from the polymorphisms in Table A.

The present invention provides nucleic acid molecules capable of detecting insertion/deletion polymorphisms (INDELs) in Arabidopsis at an average density of one INDEL per 8.4 kb. The present invention also provides nucleic acid molecules capable of detecting single nucleotide polymorphisms (SNPs) at an average density of one SNP per 3.9 kb. The present invention also provides nucleic acid molecules capable of detecting polymorphisms at an average density of one polymorphism per 2.7 kb.

As used herein, an “INDEL” is any insertion/deletion polymorphism characterized by additional nucleotides in at least one allele as compared to a reference allele. As used herein, a “SNP” is any polymorphism characterized by a different single nucleotide at a particular physical position in at least one allele.

The polymorphisms capable of detection by nucleic acid molecules of the present invention are distributed throughout the genome of the mapping population in a manner that allows the efficient identification of a genomic region associated with a phenotypic trait. In a preferred embodiment, the polymorphisms are distributed throughout the genome where 60%, preferably 70%, more preferably 80%, even more preferably 90%, 95% or 100% of the genome has a characterized polymorphism at a density of higher than one polymorphism per 100 kb, more preferably higher than one polymorphism per 50 kb, and even more preferably higher than one polymorphism per 25 kb, 10 kb, 7 kb, 5 kb or 3 kb. In another preferred embodiment, the polymorphisms are distributed throughout the genome where 60%, preferably 70%, more preferably 80%, even more preferably 90%, 95% or 100% of genome has a characterized polymorphism at a density of higher than one polymorphism per 3.5 cM, more preferably higher than one polymorphism per 3.25 cM, and even more preferably higher than one polymorphism per 3.0 cM, 2.75 cM, 2.5 cM, 2.0 cM, 1.5 cM, 1.0 cM or 0.5 cM.

In a preferred embodiment of the present invention, the efficient identification of a genomic region associated with a phenotypic trait, e.g. a QTL or a single gene, is provided, where the genomic region is less than 100 kb, more preferably less than 50 kb, and even more preferably less than 25 kb, 10 kb, 7 kb, 5 kb or 3 kb from a characterized polymorphism. In another preferred embodiment of the present invention the efficient identification of a genomic region associated with a phenotypic trait where the genomic region is less than 3.5 cM, more preferably less than 3.25 cM, and even more preferably less than 3 cM, 2.75 cM, 2.5 cM, 2.0 cM, 1.5 cM, 1.0 cM or 0.5 cM from a characterized polymorphism.

It is understood that the distribution of polymorphisms need not be uniform in a genome as certain regions will exhibit a higher average density of polymorphisms (e.g. non-centromeric regions) and certain regions will exhibit a lower average density of polymorphisms (e.g. centromeric regions).

In a preferred embodiment, the efficient identification of a genomic region associated with a phenotypic trait of interest will be obtained by a simultaneous screening for the presence of 25 or more, more preferably 50 or more, even more preferably 75 or more, 100 or more, 150 or more, 200 or more, 250 or more, 300 or more, 400 or more or 500 or more, 1,000 or more, 2,000 or more, 3,000 or more, 4,000 or more polymorphisms. When high throughput assays are emploued, e.g. with microarrays, it may be feasible to screen for the presence of 5,000 or more polymorphisms, e.g. at least 10,000 or even 15,000 polymorphisms. In an even more preferred embodiment, the efficient identification of a genomic region associated with a phenotypic trait of interest will be obtained by a simultaneously screening for the presence of 25 or more, more preferably 50 or more, even more preferably (where appropriate) 100 or more, or 250 or more etc. of the polymorphisms in Table A.

In another preferred embodiment, the efficient identification of a genomic region associated with a phenotypic trait of interest will be obtained by screening for the presence of 25 or more, more preferably 50 or more, even more preferably 75 or more, 100 or more, 150 or more, 200 or more, 250 or more, 300 or more, 400 or more or 500 or more, 1,000 or more, 2,000 or more, 3,000 or more, 4,000 or more polymorphisms during a single assay. In an even more preferred embodiment the efficient identification of a genomic region associated with a phenotypic trait of interest will be obtained by screening for the presence of 25 or more, more preferably 50 or more, even more preferably (where appropriate) 100 or more or 250 or more etc. of the polymorphisms in Table A during a single assay. A single assay can comprise many steps. One or more of these steps can occur sequentially.

In an embodiment of the present invention, the assay is carried out using a high throughput system. A particularly preferred high throughput system involves a solid phase array. A particularly preferred solid phase array is a microarray.

In the assays below, a collection of markers for polymorphisms can comprise from a few up to millions of different nucleic acid molecules. For example, using simple dot-blot hybridization methods, membranes with many nucleic acid molecules can be generated for screening. The solid-phase techniques described below and known in the art can be adapted for high-throughput monitoring of polymorphisms. In such methods different immobilized nucleic acid molecule probes can be placed on a solid support at microarray densities of up to millions of nucleic acid molecules per square inch. Similarly, very large sets of nucleic acid molecules can be immobilized for simultaneous screening against one or more probes.

Several methods have been described for fabricating microarrays of nucleic acid molecules and using such microarrays in detecting nucleic acid sequences. For instance, microarrays of markers for polymorphisms can be fabricated by spotting nucleic acid molecules, e.g. oligonucleotides, onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1,000 per square centimeter or higher. Useful substrates for arrays include nylon, glass and silicon. See, for instance, U.S. Pat. Nos. 5,202,231; 5,242,974; 5,384,261; 5,405,783; 5,412,087; 5,424,186; 5,429,807; 5,436,327; 5,445,934; 5,472,672; 5,525,464; 5,527,681; 5,529,756; 5,532,128; 5,545,531; 5,554,501; 5,556,752; 5,561,071; 5,571,639; 5,593,839; 5,599,695; 5,624,711; 5,658,734; 5,700,637; 5,744,305; 5,800,992; 5,807,522; 6,004,755 and 6,087,102 the disclosures of all of which are incorporated herein by reference in their entireties.

Sequences can be efficiently analyzed by hybridization or primer extension. See, for instance, U.S. Pat. Nos. 5,202,231; 5,445,934; 5,492,806; 5,525,464; 5,695,940; 5,700,637; 5,744,305; 5,800,992; 5,807,522; and 5,830,645, all of which are incorporated herein by reference in their entirety. Nucleic acid molecule microarrays may be screened with molecules or fragments thereof to determine nucleic acid molecules that specifically bind molecules or fragments thereof.

In a preferred embodiment, a microarray of the present invention comprises at least 10 nucleic acid molecules that specifically hybridize under high stringency to at least 10 polymorphic nucleic acid sequences characterized by this invention. In a more preferred embodiment, a microarray of the present invention comprises at least 100 nucleic acid molecules that specifically hybridize under high stringency to at least 100 characterized polymorphic nucleic acid sequences; more preferably at least 1,000 or 2,500 marker nucleic acid molecules that specifically hybridize under high stringency to at least 1,000 or 2,500 characterized polymorphic nucleic acid sequences; even more preferably at least at least 4,000 or more marker nucleic acid molecules that specifically hybridize under high stringency to at least 4,000 or more characterized polymorphic nucleic acid sequences.

In a preferred embodiment, a microarray of the present invention comprises at least 10 nucleic acid molecules capable of detecting or characterizing by primer extension to at least 10 polymorphic nucleic acid sequences characterized by this invention. In a more preferred embodiment, a microarray of the present invention comprises at least 100 nucleic acid molecules capable of detecting or characterizing by primer extension to at least 100 characterized polymorphic nucleic acid sequences; even more preferably at least 1,000 or 2,500 nucleic acid molecules capable of detecting or characterizing by primer extension to at least 1,000 or 2,500 characterized polymorphic nucleic acid sequences; even more preferably at least 4,000 or more nucleic acid molecules capable of detecting or characterizing by primer extension to at least 4,000 or more characterized polymorphic nucleic acid sequences.

In a preferred embodiment, the microarray is a variant detector array (VDA)(Cho et al., Nature Genetics 23:203-207 (1999); Wang et al., Science 280: 1077-1082 (1998), the entirety of which is herein incorporated by reference; Winzeler et al., Curr. Opin. Genet. Dev. 4: 602-608 (1997), the entirety of which is herein incorporated by reference). For example, each detection block can consist of four variant detector arrays (VDAs) corresponding to the alternative alleles: two for the forward strand sequence and two for the reverse strand sequence (See e.g. Cho et al., Nature Genetics 23:203-207 (1999)). For each of the interrogated positions (for example, −5 to +5 relative to the polymorphic position), a set of four suitable length oligonucleotides per SNP or other polymorphism (e.g. 25-mers are prepared where the oligonucleotides are complementary to the SNP or other polymorphic region except at the interrogated position). Hybridization of the oligonucleotides with the matching allele results in a strong signal.

The detection or screening of polymorphisms in a sample of DNA may be facilitated, for example, through including the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means. For instance, polymorphisms in DNA sequences can be detected by a variety of effective methods well known in the art including those disclosed in U.S. Pat. Nos. 5,468,613 and 5,217,863; 5,210,015; 5,876,930; 6,030,787 6,004,744; 6,013,431; 5,595,890; 5,762,876; 5,945,283; 5,468,613; 6,090,558; 5,800,944 and 5,616,464, all of which are incorporated herein by reference in their entireties. In particular, polymorphisms in DNA sequences can be detected by hybridization to allele-specific oligonucleotide (ASO) probes as disclosed in U.S. Pat. Nos. 5,468,613 and 5,217,863. The nucleotide sequence of an ASO probe is designed to form either a perfectly matched hybrid or to contain a mismatched base pair at the site of the variable nucleotide residues. The distinction between a matched and a mismatched hybrid is based on differences in the thermal stability of the hybrids in the conditions used during hybridization or washing, differences in the stability of the hybrids analyzed by denaturing gradient electrophoresis or chemical cleavage at the site of the mismatch.

If a polymorphism creates or destroys a restriction endonuclease cleavage site, or if it results in the loss or insertion of DNA (e.g., a Variable Number of Tandem Repeats (VNTR) polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”). RFLPs have been widely used in human and plant genetic analyses (Glassberg, UK Patent Application 2135774; Skolnick et al., Cytogen. Cell Genet. 32:58-67 (1982); Botstein et al., Ann. J. Hum. Genet. 32:314-331 (1980); Fischer et al., PCT Application WO 90/13668; Uhlen, PCT Application WO 90/11369, all of which are herein incorporated by reference in their entirety).

An alternative method of determining polymorphisms is based on cleaved amplified polymorphic sequences (CAPS) (Konieczny, A. and F. M. Ausubel, Plant J. 4:403-410 (1993); Lyamichev et al., Science 260:778-783 (1993), the entireties of which are herein incorporated by reference). One advantage of this method is the large amount of target DNA that is generated by amplification which elimiates the requirement for radiolabeling for detection of the polymorphism.

Polymorphisms can also be identified by single strand conformation polymorphism (SSCP) analysis. The SSCP technique is a method capable of identifying most sequence variations in a single strand of DNA, typically between 150 and 250 nucleotides in length (Elles, Methods in Molecular Medicine: Molecular Diagnosis of Genetic Diseases, Humana Press (1996), the entirety of which is herein incorporated by reference; Orita et al., Genomics 5:874-879 (1989), the entirety of which is herein incorporated by reference). Under denaturing conditions a single strand of DNA will adopt a conformation that is uniqely dependent on its sequence. This conformation ususally will be different even if only a single base is changed. Most conformations have been reported to alter the physical configuration or size sufficiently to be detectable by electrophoresis. A number of protocols have been described for SSCP including, but not limited to Lee et al., Anal. Biochem. 205:289-293 (1992); the entirety of which is herein incorporated by reference; Suzuki et al., Anal. Biochem. 192:82-84 (1991), the entirety of which is herein incorporated by reference; Lo et al., Nucleic Acids Research 20:1005-1009 (1992), the entirety of which is herein incorporated by reference; Sarkar et al., Genomics 13:441-443 (1992), the entirety of which is herein incorporated by reference).

Polymorphisms may also be detected using a DNA fingerprinting technique called amplified fragment length polymorphism (AFLP), which is based on the selective PCR amplification of restriction fragments from a total digest of genomic DNA to profile that DNA. Vos et al., Nucleic Acids Res. 23:4407-4414 (1995), the entirety of which is herein incorporated by reference. This method allows for the specific co-amplification of many restriction fragments, which can be analyzed without knowledge of the nucleic acid sequence. AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel (Beismann et al., Mol. Ecol. 6:989-993 (1997); Janssen et al., Int. J. Syst. Bacteriol 47:1179-1187 (1997); Huys et al., Int. J. Syst. Bacteriol. 47:1165-1171 (1997); McCouch et al., Plant Mol. Biol. 35:89-99 (1997); Nandi et al., Mol. Gen. Genet. 255:1-8 (1997); Cho et al. Genome 39:373-378 (1996); Simons et al., Genomics 44:61-70 (1997); Cnops et al., Mol. Gen. Genet. 253:32-41 (1996); Thomas et al., Plant J. 8:785-794 (1995), all of which are herein incorporated by reference in their entirety).

Polymorphisms may also be detected using random amplified polymorphic DNA (RAPD) (Williams et al., Nucl. Acids Res. 18:6531-6535 (1990), the entirety of which is herein incorporated by reference).

SNPs and insertion/deletions can be detected by methods as disclosed in U.S. Pat. Nos. 5,210,015; 5,876,930 and 6,030,787 in which an oligonucleotide probe having reporter and quencher molecules is hybridized to a target polynucleotide. The probe is degraded by 5′→3′ exonuclease activity of a nucleic acid polymerase. A useful assay is available from AB Biosystems as the Taqman® assay.

Specific nucleotide variations such as SNPs and insertion/deletions can also be detected by labeled base extension methods as disclosed in U.S. Pat. Nos.6,004,744; 6,013,431; 5,595,890; 5,762,876; and 5,945,283. These methods are based on primer extension and incorporation of detectable nucleoside triphosphates. The primer is designed to anneal to the sequence immediately adjacent to the variable nucleotide which can be can be detected after incorporation of as few as one labeled nucleoside triphosphate. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

SNPs generally occur at greater frequency than other polymorphic markers and are spaced with a greater uniformity throughout a genome than other reported forms of polymorphism. The greater frequency and uniformity of SNPs means that there is greater probability that such a polymorphism will be found near or in a genetic locus of interest than would be the case for other polymorphisms. SNPs are located in protein-coding regions and noncoding regions of a genome. Some of these SNPs may result in defective or variant protein expression (e.g., as a result of mutations or defective splicing). Analysis (genotyping) of characterized SNPs can require only a plus/minus assay rather than a lengthy measurement, permitting easier automation.

Other methods for identifying and detecting SNPs include the direct or indirect sequencing of the site, the use of restriction enzymes (Botstein et al., Am. J. Hum. Genet. 32:314-331 (1980); and Konieczny and Ausubel, Plant J. 4:403-410 (1993)), enzymatic and chemical mismatch assays (Myers et al., Nature 313:495-498 (1985)), allele-specific PCR (Newton et al., Nucl. Acids Res. 17:2503-2516 (1989); and Wu et al., Proc. Natl. Acad. Sci. USA 86:2757-2760 (1989)), ligase chain reaction (Barany, Proc. Natl. Acad. Sci. USA 88:189-193 (1991)), single-strand conformation polymorphism analysis (Labrune et al., Am. J. Hum. Genet. 48: 1115-1120 (1991)), single base primer extension (Kuppuswamy et al., Proc. Natl. Acad. Sci. USA 88:1143-1147 (1991); and Goelet, U.S. Pat. Nos. 6,004,744 and 5,888,819 both of which are incorporated herein by reference), solid-phase ELISA-based oligonucleotide ligation assays (Nikiforov et al., Nucl. Acids Res. 22:4167-4175 (1994)), dideoxy fingerprinting (Sarkar et al., Genomics 13:441-443 (1992)), oligonucleotide fluorescence-quenching assays (Livak et al., PCR Methods Appl. 4:357-362 (1995)), 5′-nuclease allele-specific hybridization TaqMan™ assay (Livak et al., Nature Genet. 9:341-342 (1995)), template-directed dye-terminator incorporation (TDI) assay (Chen and Kwok, Nucl. Acids Res. 25:347-353 (1997)), allele-specific molecular beacon assay (Tyagi et al., Nature Biotech. 16: 49-53 (1998)), PinPoint assay (Haff and Smirnov, Genome Res. 7: 378-388 (1997)), dCAPS analysis (Neffetal., Plant J. 14:387-392 (1998)), pyrosequencing (Ronaghi et al, Analytical Biochemistry 267:65-71 (1999); Ronaghi et al PCT application WO 98/13523; and Nyren et al PCT application WO 98/28440), using mass spectrometry e.g., the Masscode™ system (Howbert et al WO 99/05319; Howber et al WO 97/27331), mass spectroscopy (U.S. Pat. No. 5,965,363, incorporated herein by reference), invasive cleavage of oligonucleotide probes (Lyamichev et al Nature Biotechnology 17:292-296), and using high density oligonucleotide arrays (Hacia et al Nature Genetics 22:164-167).

INDELs are identified by comparing sequence of Arabidopsis thaliana ecotypes Columbia and Landsberg erecta. Certain INDELs are believed to have resulted from insertion or excision of transposable elements. Thus, INDEL sequences can be used to identify candidate sequences for active transposons by comparing INDEL sequences to the sequence of known transposons. For instance, certain INDEL sequences of greater than 100 bp were found to exhibit similarity to the sequence of MuDR transposable element from maize.

Polymorphisms may also be detected using allele-specific oligonucleotides (ASO), which, can be for example, used in combination with hybridization based technology including southern, northern, and dot blot hybridizations, reverse dot blot hybridizations and hybridizations performed on microarray and related technology.

The stringency of hybridization for polymorphism detection is highly dependent upon a variety of factors, including length of the allele-specific oligonucleotide, sequence composition, degree of complementarity (i.e. presence or absence of base mismatches), concentration of salts and other factors such as formamide, and temperature. These factors are important both during the hybridization itself and during subsequent washes performed to remove target polynucleotide that is not specifically hybridized. In practice, the conditions of the final, most stringent wash are most critical. In addition, the amount of target polynucleotide that is able to hybridize to the allele-specific oligonucleotide is also governed by such factors as the concentration of both the ASO and the target polynucleotide, the presence and concentration of factors that act to “tie up” water molecules, so as to effectively concentrate the reagents (e.g., PEG, dextran, dextran sulfate, etc.), whether the nucleic acids are immobilized or in solution, and the duration of hybridization and washing steps.

Hybridizations are preferably performed below the melting temperature (Tₘ) of the ASO. The closer the hybridization and/or washing step is to the Tₘ, the higher the stringency. Tₘ for an oligonucleotide may be approximated, for example, according to the following formula: Tₘ=81.5+16.6×(log 10[Na+])+0.41*(% G+C)−675/n; where [Na+] is the molar salt concentration of Na+ or any other suitable cation and n=number of bases in the oligonucleotide. Other formulas for approximating Tₘ are available and are known to those of ordinary skill in the art.

Stringency is preferably adjusted so as to allow a given ASO to differentially hybridize to a target polynucleotide of the correct allele and a target polynucleotide of the incorrect allele. Preferably, there will be at least a two-fold differential between the signal produced by the ASO hybridizing to a target polynucleotide of the correct allele and the level of the signal produced by the ASO cross-hybridizing to a target polynucleotide of the incorrect allele (e.g., an ASO specific for a mutant allele cross-hybridizing to a wild-type allele). In more preferred embodiments of the present invention, there is at least a five-fold signal differential. In highly preferred embodiments of the present invention, there is at least an order of magnitude signal differential between the ASO hybridizing to a target polynucleotide of the correct allele and the level of the signal produced by the ASO cross-hybridizing to a target polynucleotide of the incorrect allele.

While certain methods for detecting polymorphisms are described herein, other detection methodologies may be utilized. For example, additional methodologies are known and set forth, in Birren et. al., Genome Analysis, 4:135-186, A Laboratory Manual. Mapping Genomes, Cold Spring Harbor Laboratory Press, Cold Spring Harbor, N.Y. (1999); Maliga et al., Methods in Plant Molecular Biology. A Laboratory Course Manual, Cold Spring Harbor Laboratory Press, Cold Spring Harbor, N.Y. (1995); Paterson, Biotechnology Intelligence Unit: Genome Mapping in Plants, R.G. Landes Co., Georgetown, Tex., and Academic Press, San Diego, Calif. (1996); The Maize Handbook, Freeling and Walbot, eds., Springer-Verlag, New York, N.Y. (1994); Methods in Molecular Medicine: Molecular Diagnosis of Genetic Diseases, Elles, ed., Humana Press, Totowa, N.J. (1996); Clark, ed., Plant Molecular Biology: A Laboratory Manual, Clark, ed., Springer-Verlag, Berlin, Germany (1997), all of which are herein incorporated by reference in their entirety.

Detection of one or more of the polymorphisms, preferably one or more of the characterized polymorphisms, may be carried out using a collection of nucleic acid markers.

Preferred aspects of this invention comprise collections of nucleic acid markers comprising nucleic acid molecules where the collections range in size from about 10 non-identical members or more, to at least about 100 or 200 or higher, more preferably at least about 300 or 350, most preferably at least 400 or 500 or higher, up to about 1,000, or 2000 or even higher, say about 4,000 or greater, or more non-identical members. High density collections of 5,000 or more polymorphism, e.g. at least 10,000 or even 15,000 polymorphisms may be useful for high throughput assays, e.g. with microarrays. As used herein a non-identical member is a member that differs in nucleic acid or amino acid sequence. For example, a non-identical nucleic acid molecule is a nucleic acid molecule that differs in nucleic acid sequence from the nucleic acid molecule to which it is being compared. For example a nucleic acid molecule having the sequence 5′ CCC 3′ is not identical—i.e. is non-identical—to a nucleic acid molecule having the sequence 5═ CCG 3′. In one limited aspect a collection may comprise all of the nucleic acid markers identified by this invention. Collections of nucleic acid markers can be located or organized in a variety of forms, e.g. on microarrays, in solutions, in bacterial clone libraries, etc. As used herein, an “organized” collection is a collection where the nucleic acid or amino acid sequence of a member of such a collection can be determined based on its physical location.

In order to simultaneously screen for multiple polymorphisms, the nucleic acid markers can be designed for simultaneous use known as multiplexing. Examples of design approaches for multiplexing are set forth in Cho et al., Nature Genetics 23:203-207 (1999); Wang et al., Science 280: 1077-1082 (1998), the entirety of which is herein incorporated by reference; Winzeler et al., Curr. Opin. Genet. Dev. 4: 602-608 (1997), the entirety of which is herein incorporated by reference. Examples of nucleic acid markers that have been optimized for multiplexing are the primers set forth in Table B. Multiplex parameters often require the selection of loci with similar amplification efficiencies, minimizing the concentration of the primers used, and an increased magnesium concentration (Cho et al., Nature Genetics 23:203-207 (1999)).

In a preferred embodiment, the polymorphism is present and screened for in a mapping population, e.g. a collection of plants capable of being used with markers such as polymorphic markers to map genetic position of traits. The choice of appropriate mapping population often depends on the type of marker systems employed (Tanksley et al., J P. Gustafson and R. Appels (eds.). Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adapted x exotic) and generally yield greatly reduced linkage distances. Wide crosses will usually provide segregating populations with a relatively large number of polymorphisms when compared to progeny in a narrow cross (adapted x adapted).

An F₂ population is the first generation of selfing (self-pollinating) after the hybrid seed is produced. Usually a single F₁ plant is selfed to generate a population segregating for all the genes in Mendelian (1:2:1) pattern. Maximum genetic information is obtained from a completely classified F₂ population using a codominant marker system (Mather, Measurement of Linkage in Heredity: Methuen and Co., (1938), the entirety of which is herein incorporated by reference). In the case of dominant markers, progeny tests (e.g., F₃, BCF₂) are required to identify the heterozygotes, in order to classify the population. However, this procedure is often prohibitive because of the cost and time involved in progeny testing. Progeny testing of F₂ individuals is often used in map construction where phenotypes do not consistently reflect genotype (e.g. disease resistance) or where trait expression is controlled by a QTL. Segregation data from progeny test populations e.g., F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant markers evaluated in RIL populations provide more information per individual than either marker type in backcross populations (Reiter. Proc. Natl. Acad. Sci. (U.S.A.) 89:1477-1481 (1992), the entirety of which is herein incorporated by reference). However, as the distance between markers becomes larger (i.e., loci become more independent), the information in RIL populations decreases dramatically when compared to codominant markers.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts or mosaic of genomic regions from the donor parent. Backcross populations can be useful for mapping dominant markers if all loci in the recurrent parent are homozygous and the donor and recurrent parent have contrasting polymorphic marker alleles (Reiter et al., Proc. Natl. Acad. Sci. (U.S.A.) 89:1477-1481 (1992), the entirety of which is herein incorporated by reference). Information obtained from backcross populations using either codominant or dominant markers is less than that obtained from F₂ populations because one, rather than two, recombinant gamete is sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) (created by many backcrosses to produce a collection of individuals that is nearly identical in genetic composition except for the trait or genomic region under interrogation) can be used as a mapping population. In mapping with NILs, only a portion of the polymorphic loci is expected to map to a selected region.

Bulk segregant analysis (BSA) is a method developed for the rapid identification of linkage between markers and traits of interest (Michelmore et al., Proc. Natl. Acad. Sci. U.S.A. 88:9828-9832 (1991), the entirety of which is herein incorporated by reference). In BSA, two bulked DNA samples are drawn from a segregating population originating from a single cross. These bulks contain individuals that are identical for a particular trait (resistant or susceptible to particular disease) or genomic region but arbitrary at unlinked regions (i.e. heterozygous). Regions unlinked to the target region will not differ between the bulked samples of many individuals in BSA.

While any appropriate mapping population may be used in conjunction with this invention, in a preferred embodiment the mapping population is an Arabidopsis population, where the population was created, at least in part, by crossing two different Arabidopsis ecotypes, where one of the ecotypes has a phenotype of interest. In an even more preferred embodiment the ecotypes are Arabidopsi, thaliana, Columbia and Arabidopsis thaliana, Landsberg erecta. In another preferred embodiment, the mapping population is an Arabidopsis population, where the population was created, at least in part, by crossing two different Arabidopsis ecotypes, where one of the ecotypes has a phenotype of interest, propagating and self pollinating seeds from such a cross and selecting a collection of plants with the phenotype of interest to be the mapping population.

Classical mapping studies often utilize easily observable, visible traits instead of molecular markers. These visible traits are also known as naked eye polymorphisms. These traits can be morphological like plant height, fruit size, shape and color or physiological like disease response, photoperiod sensitivity and crop maturity. Visible traits are useful and are still in use because they represent actual phenotypes and are easy to score without any specialized lab equipment. By contrast, many nucleic acid markers are arbitrary loci for use in linkage mapping and often not associated with specific plant phenotypes (Young, Encyclopedia of Agricultural Science, Vol. 3, pp. 275-282 (1994), the entirety of which is herein incorporated by reference). Many morphological markers cause such large effects on phenotype that they are undesirable in breeding programs. Many other visible traits have the disadvantage of being developmentally regulated (i.e., expressed only at certain stages; or in specific tissue and organs). Oftentimes, visible traits mask the effects of linked minor genes making it nearly impossible to identify desirable linkages for selection (Tanksley et al., Biotech. 7:257-264 (1989), the entirety of which is herein incorporated by reference).

Although a number of important agronomic characteristics are controlled by loci having major effects on phenotype, many economically important traits, such as yield and some forms of disease resistance, are quantitative in nature. This type of phenotypic variation in a trait is typically characterized by continuous, normal distribution of phenotypic values in a particular population (polygenic traits) (Beckmann and Soller, Oxford Surveys of Plant Molecular Biology, Miffen. (ed.), Vol. 3, Oxford University Press, UK., pp. 196-250 (1986), the entirety of which is herein incorporated by reference). Loci contributing to such genetic variation are often termed minor genes, as opposed to major genes with large effects that follow a Mendelian pattern of inheritance. Polygenic traits are also predicted to follow a Mendelian type of inheritance, however the contribution of each locus is expressed as an increase or decrease in the final trait value. The nucleic acid markers of the present invention can be used to identify and isolate nucleic acid regions or molecules associated with desired polygenic or single gene traits.

In one embodiment, the nucleic acid markers of the present invention are used to isolate or identify an allele of a quantitative trait locus or Mendelian locus.

Nucleic acid markers of the present invention capable of detecting one or more of the polymorphisms may be employed in genetic or physical studies using linkage analysis. Mapping marker genetic locations is based on the observation that two markers located near each other on the same chromosome will tend to be passed together from parent to offspring. During gamete production, DNA strands occasionally break and rejoin in different places on the same chromosome or on the homologous chromosome. The closer the markers are to each other, the more tightly linked and the less likely a recombination event will fall between and separate them. Recombination frequency thus provides an estimate of the distance between two markers.

Linkage analysis is based on the level at which markers and genes are co-inherited (Rothwell, Understanding Genetics. 4^{th} Ed. Oxford University Press, New York, p. 703 (1988), the entirety of which is herein incorporated by reference). Statistical tests like chi-square analysis can be used to test the randomness of segregation or linkage (Kochert, The Rockefeller Foundation International Program on Rice Biotechnology, University of Georgia Athens, Ga., pp. 1-14 (1989), the entirety of which is herein incorporated by reference). In linkage mapping, the proportion of recombinant individuals out of the total mapping population provides the information for determining the genetic distance between the loci (Young, Encyclopedia of Agricultural Science, Vol. 3, pp. 275-282 (1994), the entirety of which is herein incorporated by reference). Any statistical analysis that establishes linkage may be used. An example of a suitable linkage approach is Intermap as set forth in Cho et al., Nature Genetics 23: 203-207 (1999). Example 6 sets forth another exemplary linkage approach.

In segregating populations, target genes have been reported to have been placed within an interval of 5-10 cM with a high degree of certainty (Tanksley et al., Trends in Genetics 11(2):63-68 (1995), the entirety of which is herein incorporated by reference). The markers defining this interval are used to screen a larger segregating population to identify individuals derived from one or more gametes containing a crossover in the given interval. Such individuals are useful in orienting other markers closer to the target gene. Once identified, these individuals can be analyzed in relation to all molecular markers within the region to identify those closest to the target.

Markers of the present invention can be employed to locate genes. The genetic linkage of additional marker molecules can be established by a genetic mapping model such as, without limitation, the flanking marker model reported by Lander and Botstein, Genetics 121:185-199 (1989), the entirety of which is herein incorporated by reference, and the interval mapping, based on maximum likelihood methods described by Lander and Botstein, Genetics 121:185-199 (1989), the entirety of which is herein incorporated by reference and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990), the entirety of which is herein incorporated by reference). Additional software includes Qgene, Version 2.23 (Department of Plant Breeding and Biometry, 266 Emerson Hall, Cornell University, Ithaca, N.Y. (1996), the manual of which is herein incorporated by reference in its entirety).

The LOD score essentially indicates how much more likely the data are to have arisen assuming the presence of an allele than in its absence. The LOD threshold value for avoiding a false positive with a given confidence, say 95%, depends on the number of markers and the length of the genome. Graphs indicating LOD thresholds are set forth in Lander and Botstein, Genetics 121:185-199 (1989), the entirety of which is herein incorporated by reference and further described by Arús and Moreno-González, Plant Breeding, Hayward, Bosemark, Romagosa (eds.) Chapman & Hall, London, pp. 314-331 (1993), the entirety of which is herein incorporated by reference.

In a preferred embodiment of the present invention the nucleic acid marker exhibits a LOD score of greater than 2.0, more preferably 2.5, even more preferably greater than 3.0 or 4.0 with the trait or phenotype of interest.

Additional models can be used. Many modifications and alternative approaches to interval mapping have been reported, including the use of non-parametric methods (Kruglyak and Lander, Genetics, 139:1421-1428 (1995), the entirety of which is herein incorporated by reference). Multiple regression methods or models can be also used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994), the entirety of which is herein incorporated by reference). Procedures combining interval mapping with regression analysis, whereby the phenotype is regressed onto a single putative QTL at a given interval, and at the same time onto a number of polymorphisms that serve as ‘cofactors,’ have been reported by Jansen and Stam, Genetics, 136:1447-1455 (1994), the entirety of which is herein incorporated by reference and Zeng, Genetics, 136:1457-1468 (1994), the entirety of which is herein incorporated by reference. Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994)), thereby improving the precision and efficiency of QTL mapping (Zeng, Genetics, 136:1457-1468 (1994), the entirety of which is herein incorporated by reference). These models can be extended to multi-environment experiments to analyze genotype-environment interactions (Jansen et al., Theo. Appl. Genet. 91:33-37 (1995), the entirety of which is herein incorporated by reference).

The nucleic acid markers of the present invention may be used to isolate an allele, a region of genomic DNA associated with a phenotype, etc. Once the genomic region associated with the phenotype of interest is defined relative to at least one nucleic acid marker, preferably at least two nucleic acid markers capable of detecting different polymorphisms, the genomic region associated with the phenotype may be further characterized. One approach is to select additional nucleic acid markers from the genomic region associated with the trait and localize the genomic region associated with the phenotype to a smaller genomic region by a technique such as fine mapping.

For example, in a preferred embodiment a method for identifying or isolating a genomic region associated with a phenotypic trait that comprises (A) screening a mapping population of Arabidopsis plants to determine the linkage of the phenotypic trait with a first collection of polymorphisms, wherein the first collection of polymorphisms is distributed throughout the genome of the mapping population of Arabidopsis plants at an average density of more than one polymorphism per about 500 kb-100 kb; (B) calculating the linkage of each of the first collection of polymorphisms to the phenotypic trait; (C) identifying a genomic region most closely associated with the phenotypic trait; (D) selecting a second collection of polymorphisms from the genomic region; and (E) screening the mapping population of Arabidopsis plants to determine the linkage of the phenotypic trait with the second collection of polymorphisms from the genomic region, wherein the second collection of polymorphisms have an average density of more than one polymorphism per about 50 kb-1 kb.

In an embodiment of the present invention, for a fine mapping step of the present invention the collection of marker nucleic acids is capable of detecting a characterized polymorphism at a density of greater than one polymorphism per 50 kb, more preferably at a density greater than one polymorphism per 25 kb, even more preferably at a density greater than one polymorphism per 10 kb or 5 kb. It is understood, that the fine mapping using such a collection of markers may be carried out, for example, in a single assay or simultaneously.

Once the genomic region associated with the phenotype is identified, the genomic region may be isolated. Alternatively, or in conjunction, such a region may be further defined or characterized. Many approaches are known in the art and may be undertaken (Sambrook et al., Molecular Cloning 1: A Laboratory Manual, 2d ed., Ford et al., eds., Cold Spring Harbor Laboratory Press, Cold Spring Harbor, N.Y. (1989); Sambrook et al., Molecular Cloning 2: A Laboratory Manual, 2d ed., Ford et al., eds., Cold Spring Harbor Laboratory Press, Cold Spring Harbor, N.Y. (1989); Sambrook et al., Molecular Cloning 3: A Laboratory Manual, 2d ed., Ford et al., eds., Cold Spring Harbor Laboratory Press, Cold Spring Harbor, N.Y. (1989); Maliga et al., Methods in Plant Molecular Biology: A Laboratory Course Manual, Cold Spring Harbor Laboratory Press, Cold Spring Harbor, N.Y. (1995); and Birren et al., Genome Analysis: A Laboratory Manual. Volume 2: Detecting Genes, Cold Spring Harbor Laboratory Press, Cold Spring Harbor, N.Y. (1998), all of which are herein incorporated by reference in their entirety). For example, once identified, the sequence of the genomic region associated with the phenotype may be determined and subjected to bioinformatic analysis (Coulson, Trends in Biotechnology 12:76-80 (1994); Birren et al., Genome Analysis 1, Cold Spring Harbor Laboratory Press, Cold Spring Harbor, N.Y. 543-559 (1997); Huang, et al., Genomics 46:37-45 (1997), all of which are herein incorporated by reference in their entirety). Such bioinformatic approaches can provide, for example, information on the location of putative open reading frames, promoters, and a variety of nucleotide motifs. Moreover, also using bioinformatic approaches, the nucleic acid sequence of the genomic region can be compared with other nucleic acid sequences. Such comparisons can facilitate the isolation of Arabidopsis homologs to known genes or genomic regions. Examples of such bioinformation tools are BLAST, GeneScan, GeneMark and AAT.

Other methods can be utilized to further isolate, define, or characterize the genomic region associated with the phenotype. The expression profiles of mRNA and proteins derived from genes that are located within the genetic region associated with the phenotype can be analyzed. Such analysis, will in certain circumstances, allow the gene or genes associated with the phenotype to be determined.

A genomic region or sub-region thereof may be isolated using any of the many techniques in the art. In addition to those procedures and methods set forth herein, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see, for example, Sambrook et al., Molecular Cloning: A Laboratory Manual, Cold Spring Harbor Press (1989); Mailga et al., Methods in Plant Molecular Biology, Cold Spring Harbor Press (1995); Birren et al., Genome Analysis: Analyzing DNA, 1, Cold Spring Harbor, N.Y., all of which are herein incorporated by reference in their entirety).

The biological function of a genomic region or subregion thereof such as a gene or open reading frame, can be further investigated using a mutant complementation approach or other reverse genetics approach. For example, a gene or genes identified within the genomic region associated with the phenotype may be isolated from the organism exhibiting the non-mutant phenotype (often referred to as the wild type). Such a gene or genes may be introduced into an appropriate organism that lacks the phenotype (often referred to as mutant) either by crosses or by molecular genetic techniques such as transformation or transfection. Organisms having the introduced genetic material may be screened to determine whether the introduced gene or genes complements, i.e. restores the phenotype of the mutant (Pan, FEBS Lett. 459(3): 405-410 (1999); Kerckhoffs et al., Mol. Gen. Genet. 6: 901-907 (1999); Lizotte et al., Gene 234(1): 35-44 (1999); Berna et al., Genetics 152: 729-742 (1999); Liu et al., Proc. Natl. Acad. Sci. (USA) 96(11): 6535-6540 (1999); Pia et al., Plant Physiol. 119(4): 1527-1534 (1999); Loulergue et al., Gene 225(1-2): 47-57 (1998); Jouannic et al., Eur. J. Bioche, 258(2): 402-410 (1998), all of which are herein incorporated by reference in their entirety). While gene or genes etc. may be introduced into any organism, preferred organisms are plants, yeasts, and bacteria particularly E. coli. In a more preferred embodiment the organism is Arabidopsis.

The nucleic acid markers of the present invention may be used for chromosomal walking. Such walking, in conjunction with linkage analysis, can enable the isolation of genes. Once a nucleic acid marker is linked to a region of interest, the chromosome walking technique can be used to find the genes via overlapping clones. For chromosome walking, random molecular markers or established molecular linkage maps are used to conduct a search to localize the gene adjacent to one or more markers capable of detecting a polymorphism. A chromosome walk (Bukanov and Berg, Mo. Microbiol. 11:509-523 (1994), the entirety of which is herein incorporated by reference; Birkenbihl and Vielmetter, Nucleic Acids Res. 17:5057-5069 (1989), the entirety of which is herein incorporated by reference; Wenzel and Herrmann, Nucleic Acids Res. 16:8323-8336, (1988), the entirety of which is herein incorporated by reference) is then initiated from the closest linked marker. Starting from the selected clones, labeled probes specific for the ends of the insert DNA are synthesized and used as probes in hybridizations against a representative library. Clones hybridizing with one of the probes are picked and serve as templates for the synthesis of new probes; by subsequent analysis, contigs are produced.

The degree of overlap of the hybridizing clones used to produce a contig can be determined by comparative restriction analysis. Comparative restriction analysis can be carried out in different ways all of which exploit the same principle; two clones of a library are very likely to overlap if they contain a limited number of restriction sites for one or more restriction endonucleases located at the same distance from each other. The most frequently used procedures are, fingerprinting (Coulson et al., Proc. Natl. Acad. Sci. (U.S.A.) 83:7821-7821, (1986), the entirety of which is herein incorporated by reference; Knott et al., Nucleic Acids Res. 16:2601-2612 (1988), the entirety of which is herein incorporated by reference; Eiglmeier et al. Mol. Microbiol. 7:197-206 (1993), the entirety of which is herein incorporated by reference), restriction fragment mapping (Smith and Birnstiel, Nucleic Acids Res. 3:2387-2398 (1976), the entirety of which is herein incorporated by reference), and the “landmarking” technique (Charlebois et al. J. Mol. Biol. 222:509-524 (1991), the entirety of which is herein incorporated by reference).

It is understood that the nucleic acid molecules of the present invention may in one embodiment be used for chromosomal walking. In a preferred embodiment, nucleic acid molecules of the present invention may in one embodiment be used in the chromosomal walking of Brassicaceae, particularly Arabidopsis thaliana.

Nucleic acid markers of the present invention can be used in comparative mapping and comparative chromosomal walking. Comparative mapping within families provides a method to assess the degree of sequence conservation, gene order, ploidy of species, ancestral relationships and the rates at which individual genomes are evolving. It also provides a method to isolate genetic regions or sub-aspects thereof such as genes. Comparative mapping has been carried out by utilizing molecular markers from one species with another species. As in genetic mapping, nucleic acid markers are needed but instead of direct hybridization to mapping filters, the markers can also be used to select large insert clones from a total genomic DNA library of a related species. The selected clones can then be used to physically map the region in the target species. The advantage of this method for comparative mapping is that no mapping population or linkage map of the target species is needed and the clones may also be used in other closely related species. By comparing the results obtained by genetic mapping in model plants, with those from other species, similarities of genomic structure among plants species can be established. Comparative mapping using nucleic acid markers of the present invention permits the identification and/or isolation of non-Arabidopsis syntenic regions and homolog genes with such regions.

It is understood that nucleic acid markers of the present invention may in another embodiment be used in comparative mapping. In a preferred embodiment the markers of the present invention may be used in the comparative mapping of non-Arabidopsis plant species, including but not limited to alfalfa, barley, Brassica, broccoli, cabbage, citrus, cotton, garlic, oat, oilseed rape, onion, canola, flax, an ornamental plant, maize, pea, peanut, pepper, potato, rice, rye, sorghum, soybean, strawberry, sugarcane, sugarbeet, tomato, wheat, poplar, pine, fir, eucalyptus, apple, lettuce, lentils, grape, banana, tea, turf grasses, sunflower, oil palm, Phaseolus etc. Particularly preferred non-Arabidopsis plants to utilize for comparative mapping are the Brassicaceae, e.g. oilseed rape.

Agents of the present invention include nucleic acid molecules and more specifically include nucleic acid markers capable of detecting polymorphisms. In a preferred embodiment the nucleic acid molecules of the present invention are derived from Arabidopsis and in an even more preferred embodiment the nucleic acid molecules of the present invention are derived from Arabidopsis thaliana, Landsberg erecta or Arabidopsis thaliana, Columbia.

In another preferred embodiment, the nucleic acid molecules of the present invention include those isolated utilizing the nucleic acid markers of the present invention. The present invention also encompasses the use of these and other nucleic acids of the present invention in recombinant constructs. Using methods known to those of ordinary skill in the art, such molecules can be introduced into a host cell or organism of choice. Potential host cells include both prokaryotic and eukaryotic cells. A host cell may be unicellular or found in a multicellular differentiated or undifferentiated organism depending upon the intended use. It is understood that useful exogenous genetic material may be introduced into any cell or organism such as a plant cell, plant, mammalian cell, mammal, fish cell, fish, bird cell, bird or bacterial cell.

In a preferred embodiment the exogenous DNA is introduced into a plant in a suitable construct. Preferred plants are selected from the group consisting of: alfalfa, Arabidopsis, barley, Brassica, broccoli, cabbage, citrus, cotton, garlic, oat, oilseed rape, onion, canola, flax, an ornamental plant, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, soybean, and Phaseolus. A particularly preferred group of plants is rice, cotton, wheat, maize and soybean.

As used herein, an agent, be it a naturally occurring molecule or otherwise may be “substantially purified,” if, referring to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

The agents of the present invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic, and thus involve the capacity of the agent to mediate a chemical reaction or response.

The agents of the present invention may can also be recombinant. As used herein, the term recombinant describes (a) nucleic acid molecules that are constructed or modified outside of cells and that can replicate or function in a living cell, (b) molecules that result from the transcription, replication or translation of recombinant nucleic acid molecules, or (c) organisms that contain recombinant nucleic acid molecules or are modified using recombinant nucleic acid molecules.

It is understood that the agents of the present invention may be labeled with reagents that facilitate detection of the agent (e.g. fluorescent labels, Prober et al., Science 238:336-340 (1987); Albarella et al., EP 144914, chemical labels, Sheldon et al., U.S. Pat. No. 4,582,789; Albarella et al., U.S. Pat. No. 4,563,417, modified bases, Miyoshi et al., EP 119448, all of which are herein incorporated by reference in their entirety).

Fragment nucleic acid molecules may encode significant portion(s) of, or indeed most of, these nucleic acid molecules. For example, a fragment nucleic acid molecule can encode an Arabidopsis protein or fragment thereof. Alternatively, the fragments may comprise smaller oligonucleotides. Exemplary fragment sizes include fragments having from about 15 to about 400 nucleotide residues and more preferably, about 15 to about 30 nucleotide residues, or about 50 to about 100 nucleotide residues, or about 100 to about 200 nucleotide residues, or about 200 to about 400 nucleotide residues, or about 275 to about 350 nucleotide residues.

Nucleic acid molecules or fragments thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989), and by Haymes et al. Nucleic Acid Hybridization, A Practical Approach, IRL Press, Washington, D.C. (1985), the entirety of which is herein incorporated by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6, the entirety of which is herein incorporated by reference. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Hybridizations involving at least one oligonucleotide can necessitate changes from the above hybridization conditions. Highly stringent conditions are often selected to be equal to the Tₘ point for a particular probe. Sometimes the term “T_{d}” is used to define the temperature at which at least half of the probe dissociates from a perfectly matched target nucleic acid. In any case, a variety of estimation techniques for estimating the Tₘ or T_{d} are available, and generally described in Tijssen, id. Typically, G-C base pairs in a duplex are estimated to contribute about 3° C. to the Tₘ, while A-T base pairs are estimated to contribute about 2° C., up to a theoretical maximum of about 80-100° C. However, more sophisticated models of T_{M} and T_{d} are available and appropriate in which G-C stacking interactions, solvent effects, the desired assay temperature and the like are taken into account. For example, PCR primers can be designed to have a dissociation temperature (T_{d}) of approximately 60° C., using the formula: T_{d}=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the primer to the template DNA.

Nucleic acid markers of the present invention can be used to characterize transformants or germplasm, as a genetic diagnostic test for plant breeding or to identify individuals or varieties (Soller and Beckmann, Theor. Appl. Genet. (67):25-33 (1983), the entirety of which is herein incorporated by reference). Such markers can also be used to obtain information about: (1) the number, effect, and chromosomal location of each gene affecting a trait; (2) effects of multiple copies of individual genes (gene dosage); (3) interaction between/among genes controlling a trait (epistasis); (4) whether individual genes affect more than one trait (pleiotropy); and (5) stability of gene function across environments (Gx E interactions).

In a preferred embodiment, the nucleic acid markers of the present invention may be used in marker assisted introgression of traits into plants. Marker assisted introgression involves the transfer of a chromosome region defined by one or more markers from one germplasm to a second germplasm. An initial step in such a process is the localization of the trait or region by mapping. One use of marker assisted introgression of genomic regions is in the generation of near isogenic lines (NILs) or recombinant near isogenic lines (RILs). In one aspect of the present invention, the nucleic acid markers are used to generate Arabidopsis NILs or RILs. As used herein, introgression is the process of transferring a genetic region from one genetic background to a second but non-identical genetic background.

Additional markers, such as AFLP markers, RFLP markers, RAPD markers, SNPs, phenotypic markers, isozyme markers can be utilized in combination with or separately from the markers of the invention (Walton, Seed World 22-29 (1993), the entirety of which is herein incorporated by reference; Burow and Blake, Molecular Dissection of Complex Traits, 13-29, Eds. Paterson, CRC Press, New York (1988), the entirety of which is herein incorporated by reference). Examples of additional markers are set forth in Cho et al., Nature Genetics 23: 203-205 (1999).

DNA markers can be developed from nucleic acid molecules using restriction endonucleases, the PCR and/or DNA sequence information. RFLP can result from single base changes or insertions/deletions. RFLP are highly abundant in plant genomes, have a medium level of polymorphism and are developed by a combination of restriction endonuclease digestion and Southern blotting hybridization. CAPS are similarly developed from restriction nuclease digestion but only of specific PCR products. CAPS are also codominant, have a medium level of polymorphism and are highly abundant in the genome. The CAPS result from single base changes and insertions/deletions. RAPDs are developed from DNA amplification with random primers and result from single base changes and insertions/deletions in plant genomes. RAPDs with a medium level of polymorphisms are highly abundant. AFLP markers require using the PCR on a subset of restriction fragments from extended adapter primers. AFLPs are both dominant and codominant are highly abundant in genomes and exhibit a medium level of polymorphism. SSRs require DNA sequence information. SSRs result from repeat length changes, are highly polymorphic, and do not exhibit as high a degree of abundance in the genome as CAPS, AFLPs and RAPDs. SNPs also require DNA sequence information. SNPs result from single base substitutions. They are highly abundant and exhibit a medium of polymorphism (Rafalski et al., In: Nonmammalian Genomic Analysis, ed. Birren and Lai, Academic Press, San Diego, Calif., pp. 75-134 (1996), the entirety of which is herein incorporated by reference).

### Computer Readable Media

A polymorphism or nucleic acid molecule of the present invention can be “provided” in a variety of mediums to facilitate use. Moreover, the nucleic acid markers and other nucleic acid molecules of the present invention may also be so presented.

In one embodiment, a polymorphism may be presented in a manner that sets forth 1, more preferably 2, 3, 4, 5, 6, or 7 of the following features alone or in combination with other features: (1) type of polymorphism (e.g. SNP, insertion, deletion etc.); (2) physical location of the polymorphism on a chromosome; (3) nucleotide sequence variation associated with one or more of the alleles; (4) nucleotide sequences of nucleic acid marker molecules capable of detecting the polymorphism; (5) physical location of the polymorphism relative to a piece of isolated DNA (e.g., BAC); (6) methodology for detecting the polymorphism; (7) physical distance from that polymorphism to another polymorphism; and (8) genetic linkage with a phenotype or other polymorphism.

Such a medium can also provide a subset thereof in a form that allows a skilled artisan to examine these features.

In one application of this embodiment, a polymorphism and associated features of the present invention can be recorded on computer readable media. In another embodiment, a nucleic acid sequence of the present invention can be recorded on computer readable media alone or in combination with a polymorphisms and associated features. As used herein, “computer readable media” refers to any medium that can be read or accessed by a computer, either directly or indirectly through a network. Such media include, but are not limited to: magnetic storage media, such as disks or magnetic tape; optical storage media such as optical disks; electrical storage media such as read-only memory (ROM) or Random Access Memory (RAM); and hybrids of these categories such as magnetic/optical storage media. A skilled artisan can readily appreciate how any of the known computer readable mediums can be used to create a manufacture comprising computer readable medium having recorded thereon a nucleotide sequence of the present invention.

As used herein, “recorded” refers to a process for storing information on computer readable medium. A skilled artisan can readily adopt any of the known methods for recording information on computer readable medium to generate media comprising the information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of application programs and formats can be used to store the information of the present invention on computer readable medium. The sequence information can be represented, for example, in a word processing file, formatted in commercially-available software such as WordPerfect and Microsoft Word, in a network-accessible format, such as an HTML file or web page, an ASCII file, or stored in a database application, such as DB2, Excel, Sybase, Oracle, or the like. A skilled artisan can readily adapt any number of data file formats (e.g., text file or database) or data structures in order to obtain computer readable medium having recorded thereon the information of the present invention.

A skilled artisan is provided with access to the information for a variety of purposes. Publicly available computer software allows a skilled artisan to access, for example, sequence information provided in a computer readable medium.

The present invention further provides systems, particularly computer-based systems, which contain the information described herein. As used herein, “a computer-based system” refers to the hardware, software, and data storage used to analyze the information including the nucleic acid sequence information of the present invention. The minimum hardware of the computer-based systems of the present invention comprises a central processing unit (CPU), input/output apparatus, and data storage. A skilled artisan can readily appreciate that any one of the currently available computer-based systems are suitable for use in the present invention.

As indicated above, the computer-based systems of the present invention comprise a data storage having stored therein a polymorphism and any associated information of the present invention and the necessary hardware and software for supporting and implementing a search. As used herein, “data storage” refers to memory that can store information of the present invention, or a memory access apparatus (hardware and/or software) that can access manufactures having recorded thereon the information of the present invention.

Having now generally described the invention, the same will be more readily understood through reference to the following examples which are provided by way of illustration, and are not intended to be limiting of the present invention, unless specified.

### EXAMPLE 1

Assembled Arabidopsis thaliana, Landsberg erecta nucleic acid sequence is generated essentially as set forth below:

DNA Preparation

DNA from Arabidopsis thaliana, Landsberg erecta seedlings is prepared by a CTAB genomic DNA isolation protocol as described by Dean et al. Plant J. 2:69-81(1992) and modified by Dubois et al. Plant J. 13:141-151 (1998), the entirety of which is herein incorporated by reference.

A solution of DNA to be sheared is prepared in a 1.5 ml microcentrifuge tube by mixing 15 μg of DNA, 6 μl of 10× mung bean (MB) buffer (10× MB buffer=300 mM NaOAc, pH 5.0, 500 mM NaCl, 10 mM ZnCl₂, 50% glycerol), and water to a final volume of 60 μl. The DNA solution is kept on ice prior to sonication. For sonication, a cup horn probe chilled with ice water for 1 hour prior to sonication is used. The sonicator (Ultrasonic Liquid Processor XL2020, Misonix Inc.) is pulsed for approximately 10 seconds on full power prior to use. DNA samples are sonicated twice for 6 seconds each at 60% power. Four sample tubes may be processed at once in a multi-tube rack which is positioned 1 to 3 mm above the opening in the probe. The DNA is returned to ice and a 1 μl sample is analyzed by electrophoresis on a 0.8% agarose gel in 0.5× TBE gel, run at 60 volts for 30 minutes. Sonication may be repeated if necessary.

A 0.26 μl aliquot of mung bean nuclease (150,000 u/ml) is added to sheared DNA and the sample is incubated at 30° C. for 10 minutes. To stop the digestion, 20 μl of 1 M NaCl, 140 μl dd H₂O, and 200 ml of phenol:chloroform are added to the sample which is then vortexed and centrifuged for 20 minutes at 13,000 rpm. The resulting aqueous phase is transferred into a new 1.5 ml microcentrifuge tube, 500 μl of 95% ethanol is added, and the DNA is precipitated overnight at −80° C. The sample is centrifuged for 30 minutes at 13,000 rpm, washed with 500 μl of 95% ethanol and centrifuged again for 30 minutes at 13,000 rpm. The sample is then dried under vacuum, and resuspended in 10 μl TE.

The sheared DNA fragments are sized and purified by preparative agarose gel electrophoresis. Five microliters of 6× BP-XC-glycerol dye (0.25% BP, 0.25% XC, 30% glycerol) is added to the sample. The sample is split into two samples and loaded (12.5 μl per lane) on a 0.8% (1× TAE) low-melting agarose gel (SeaPlaque GTG) and electrophoresed at 60 V, 46 mA for 3.5 hours.

The gel is photographed under long wave UV and slices containing DNA fragments of 1.3-1.7 kb and 2-4 kb are excised and excess agarose cut away. The gel slices are placed in 1.5 ml microcentrifuge tubes. One gel slice is stored at −20° C. 15 μl of 1 M NaCl is added to the other gel slice, followed by melting of the agarose by incubation at 65° C. for 8 minutes. The resulting approximately 250 μl samples are placed into microcentrifuge tubes. An equal volume of water is added, following which the sample is vortexed and placed at room temperature for 2 minutes to bring the temperature up to 30-35° C. 0.5 ml of water-saturated phenol that has been cooled on ice is added and the sample vortexed vigorously. The sample is placed on ice for 5 minutes, and the vortexing step repeated.

The sample is centrifuged at 4° C. in a microcentrifuge for 20 minutes. The upper phase is transferred to a clean tube, and the bottom phenol layer is reextracted by addition of 200 μl of dd H₂O. The sample is vortexed and placed on ice for 5 minutes, followed by centrifugation for 15 minutes. The aqueous layer is extracted and added to the aqueous layer from the previous step. Phenol extraction is repeated with 0.5 ml phenol, followed by vortexing and centrifugation for 20 minutes at 4° C. The aqueous layer is removed and repeated sec-butanol extractions are performed until the final volume is reduced to approximately 0.165 ml.

Two volumes of 95% ethanol (400 μl) are added and the sample is stored at −80° C. overnight. The sample is centrifuged for 30 minutes at room temperature to pellet the DNA, washed once with 95% ethanol and dried briefly under vacuum. The sample is resuspended in 7 μl of TE. A 1 μl sample is run on a 0.8% agarose gel with markers to estimate concentration of recovered fraction.

M13 Library

20 ng of M13 DNA digested with SmaI is mixed with 1 μl of 10× ligation buffer (10× ligation buffer=0.5M tris pH 7.4, 0.1M MgCl₂, 0.1M DDT), 1 μm of 1 mM ATP and 100-200 ng of sheared genomic DNA fragments (1-3 μl volume), and 0.3 μl of high concentration NEB ligase (5 unit/μl) is added. Water is added to a final volume of 10 μl and the sample is incubated overnight at 14° C.

Plasmid Library

200 ng (4 μl) of pSTBlue vector (Novegene) is mixed with approximately 600 ng (12 μl) of sheared genomic DNA fragments from the 2-4 kb size range gel slices and 1.2 μl of Gibco T4 ligase (5 units per μl) is added. Water is added to a final volume of 30 μl and the sample is incubated overnight at 14° C.

Transformation

The ligation reaction is titered and diluted for optimal transformation efficiency. When the ligation contains approximately 20 ng of M13 vector, the dilution will typically be from 1:25 to 1:100. A 1:25 dilution is used for plasmid ligation containing approximately 200 ng of vector DNA. To increase transformation efficiency, the ligase is denatured by heating at 65° C. for 7 minutes, and placed at room temperature for 5 minutes following the heating step.

A sterile electroporation cuvette is chilled for each transformation. Electro-competent cells are removed from the −80° C. freezer and thawed on ice. For each M13 transformation, a sterile tube containing 25 ul of IPTG (25 mg/ml in water), 25 μl of X-Gal (25 mg/ml in dimethylformamide) and 3 ml of YT top agar is prepared, capped and placed in a 45° C. water bath. YT plates are pre-warmed at 37° C. for several hours to avoid cross-contamination problems that may result if water remains on plates. For plasmid transformations, a sterile tube containing 0.5 ml of SOC medium is prepared for each transformation, and L+amp plates are pre-spread with 25 μl of IPTG and 25 μl of X-Gal.

25 μl of electro-competent cells are mixed with DNA in diluted ligation mix in the cuvette, and the sample pulsed in an E. coli pulser (BioRad) set to the appropriate voltage (1.80 kV for 0.1 cm cuvettes; 2.50 kV for 0.2 cm cuvettes). The cuvette is removed from the pulser, and the sample immediately transferred to the tube containing SOC or YT top agar. For M13 transfections, the sample is plated immediately on YT plates. For plasmid transformations, the tube is placed in a 37° C. shaker for 15-30 minutes and 30 ul aliquots are plated on L+Amp plates. Plates are incubated at 37° C. overnight.

Two basic methods can be used for DNA sequencing, the chain termination method of Sanger et al., Proc. Natl. Acad. Sci. (U.S.A.) 74.5463-5467 (1977), the entirety of which is herein incorporated by reference and the chemical degradation method of Maxam and Gilbert, Proc. Natl. Acad. Sci. (U.S.A.) 74:560-564 (1977), the entirety of which is herein incorporated by reference. Automation and advances in technology such as the replacement of radioisotopes with fluorescence-based sequencing have reduced the effort required to sequence DNA (Craxton, Methods 2:20-26 (1991), the entirety of which is herein incorporated by reference; Ju et al., Proc. Natl. Acad. Sci. (U.S.A.) 92:4347-4351 (1995), the entirety of which is herein incorporated by reference; Tabor and Richardson, Proc. Natl. Acad. Sci. (U.S.A.) 92:6339-6343 (1995), the entirety of which is herein incorporated by reference). Automated sequencers are available from, for example, Pharmacia Biotech, Inc., Piscataway, N.J. (Pharmacia ALF), LI-COR, Inc., Lincoln, Neb. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997), all of which are herein incorporated by reference in their entirety).

A number of sequencing techniques are known in the art, including fluorescence-based sequencing methodologies. These methods have the detection, automation and instrumentation capability necessary for the analysis of large volumes of sequence data. Currently, the 377 DNA Sequencer (Perkin-Elmer Corp., Applied Biosystems Div., Foster City, Calif.) allows the most rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA, 1, Cold Spring Harbor, N.Y., the entirety of which is herein incorporated by reference).

PHRED is used to call the bases from the sequence trace files (www.mbt.washington.edu). PHRED uses Fourier methods to examine the four base traces in the region surrounding each point in the data set in order to predict a series of evenly spaced predicted locations. That is, it determines where the peaks would be centered if there are no compressions, dropouts, or other factors shifting the peaks from their “true” locations. Next, PHRED examines each trace to find the centers of the actual, or observed peaks and the areas of these peaks relative to their neighbors. The peaks are detected independently along each of the four traces so many peaks overlap. A dynamic programming algorithm is used to match the observed peaks detected in the second step with the predicted peak locations found in the first step.

After the base calling is completed, two sequence quality steps occur 1) poor quality end sequences are cut and if the resulting sequence is 50 bp or less it is deleted 2) overall sequence quality is examined and poor sequences are deleted from the data set if they have an average quality cutoff below 12.5. Contaminating sequences (E. coli, yeast, vector, linker) are removed after sequence quality assessment.

Contigs are assembled using PANGEA clustering tools (PANGEA SYSTEMS. INC) and PHRAP (www.mbt.washington.edu). PANGEA clustering tools are a series of scripts which group sequences (clusters) by comparing pairs of sequences for overlapping bases. The overlap is determined using the following high stringency parameters: word size=8; window size=60; and identity is 93%. Each of the clusters are then assembled using PHRAP. The final assembly output contains a collection of sequences including contigs, sequences representing the consensus sequence of overlapping clustered sequences, and singletons, sequences which are not present in any cluster of related sequences. Collectively, the contigs and singletons resulting from a DNA assembly are referred to as islands.

### EXAMPLE 2

INDELs are identified by aligning sequences from Arabidopsis thaliana, Columbia and Arabidopsis thaliana, Landsberg erecta. Finished BAC sequences derived from Arabidopsis thaliana, Columbia are obtained from GenBank (www.ncbi.nlm.nih.gov/entrez/query.fcgi?db=Nucleotide). Because the GenBank sequences are subject to change, the finished sequences of the Arabidopsis thaliana, Columbia BACs are included herein as SEQ ID NO: 1 through SEQ ID NO: 1482. The sequence of each Arabidopsis thaliana, Columbia BAC is used as a query against a database of Arabidopsis thaliana, Landsberg erecta islands using the GAP2 program of the Analysis and Annotation Tool (AAT) for Finding Genes in Genomic Sequences which was developed by Xiaoqiu Huang at Michigan Tech University and is available at the web site genome.cs.mtu.edu/. See Huang, et al., Genomics 46: 37-45 (1997) and Huang, Computer Applications in the Biosciences 10 227-235 (1994), both of which are herein incorporated by reference in their entirety. The GAP2 program compares the query sequence with a cDNA database using a fast database search program and a rigorous alignment program. The database search program quickly identifies regions of the query sequence that are similar to a database sequence. Then the alignment program constructs an optimal alignment for each region and the database sequence. The output file of GAP2 is reviewed for insertions or deletions. Using alignments that are at least 96% identical (as reported by AAT), insertions and deletions are determined by looking for gaps of at least three bases, with three aligned bases on either side of the gap. To ensure that an insertion or deletion is derived from matched sequence, the 10 bp region to either side of the gap is aligned and compared. To be considered an insertion or deletion, the adjacent aligned regions must be at least 90% identical (as reported by AAT). Insertions or deletions smaller than 100 bp are considered candidate markers. INDELs identified by the method of this Example 2 are set forth in Tables A1 and A2 and identified in the “method” column by reference to method 2. More particularly Tables A1 and A2 identify the location and nature of the polymorphism as follows:

“SEQ NUM” refers to the sequence of the finished BAC of Arabidopsis thaliana, ecotype Columbia where the polymorphism can be found;

“SEQ ID” refers to an arbitrary name used by applicant to identify the BAC sequence;

“Chromosome” refers to the chromosome of Arabidopsis thaliana in which the polymorphism is located;

“BAC Length” refers to the number of nucleotides in the finished BAC sequence;

“BAC Name” refers to the name of the BAC as used in GenBank;

“Marker Name” refers to a unique six digit number arbitrarily set by applicant for a polymorphism;

“Left” refers to the position of the closest nucleotide in the flanking sequence on the 5′ side of the polymorphism;

“Right” refers to the position of the closest nucleotide in the flanking sequence on the 3′ side of the polymorphism;

“Type” refers to identification of the polymorphism as a SNP or IND (i.e., INDEL);

“Method” refers to the method used to identify the polymorphism, where “1” represents the method of Example 3 used to detect SNPs and INDELs of less than 3 nucleotides and “2” represents the method of Example 2 used to detect large INDELs; and

“Indel Size” refers to the size of INDELs in terms of “n/−n” or “−n/n”, where n is the size of the insertion or deletion and the minus sign indicates the ecotype, Columbia/Landsberg, respectively, with the smaller sequence length in the area of the polymorphism.

“Columbia/Landsberg” describes the nucleotide base of a SNP in the respective ecotypes, e.g. “T/C.”

### EXAMPLE 3

SNPs are identified by comparing Arabidopsis thaliana, Columbia and Arabidopsis thaliana, Landsberg erecta sequences. Each Arabidopsis thaliana, Columbia BAC sequence (extracted from GenBank and represented by a SEQ ID NO: 1 through SEQ ID NO: 124) is compared to a full set of Arabidopsis thaliana, Landsberg erecta contigs using WUBLAST (version 2.0) to locate areas of high identity that could contain a marker. Each identified contig is subsequently compared using WUBLAST to a full set of Arabidopsis thaliana, Columbia BACs (all of SEQ ID NO: 1 through SEQ ID NO: 124). To be selected as a marker candidate, an Arabidopsis thaliana, Landsberg erecta contig must have either one or two matches to an Arabidopsis thaliana, Columbia BAC. A single match suggests that that the sequence is unique. Two matches often result from overlapping BACs. The alignments are evaluated in a conservative manner. False negatives are preferable to false positives. To be included as a candidate polymorphic marker there must be: a minimum alignment of 200 bases between the sequence of an Arabidopsis thaliana, Landsberg erecta contig and the sequence of an Arabidopsis thaliana, Columbia BAC; the alignment must cover at least 75% of the length of the Arabidopsis thaliana, Landsberg erecta contig; a minimum of two reads of the Arabidopsis thaliana, Landsberg erecta region with the two read areas extending at least 25 bases on each side of the polymorphism position; agreement between all Arabidopsis thaliana, Landsberg erecta reads at the polymorphism position; minimum PHRAP consensus quality of 40 at the polymorphism position, with an average quality of 30 for the 25 bases on each side of the polymorphism position; and a maximum 1% polymorphism across the sequence. SNPs and INDELs of less than three nucleotide bases identified as described above are set forth in Tables A1 and A2.

A set of fifty polymorphisms was selected from among the polymorphisms in Tables A1 and A2 and identified more particularly with primer pairs in Table B as core selection markers. The primer pair oligonucletides for these core markers have the sequence of SEQ ID NO: 1483 through SEQ ID NO: 1582.

More particularly Table B identifies the primers for use with the core set of 50 polymorphisms as follows:

“SEQ NUM” refers to the SEQ ID NO for the oligonucleotide primer.

“Marker Name” refers to either an aribitrary six digit identifier or the public name from GenBank.

“Primer Name” refers identifies the primer by adding a F (for forward) or R (for reverse) after the corresponding Marker Name.

“Chromosome” refers to the Arabidopsis thaliana chromosome on which the polymorphism is located

“Genetic Distance (cM)” refers to the genetic distance of the polymorphism from the begininng of the chromosome.

“Size of Insert Col/Ler” refers to the number of base pairs inserted in listed ecotype for an INDEL polymorphism. Col identifies the Columbia ecotype and Ler identifies the Landsberg erecta ecotype.

“Amplicon Size Col/Ler” refers to the length of PCR product using the forward and reverse primers for a polymorphism for the listed ecotype.

TABLE B

| | | | | Genetic | Size of | | Amplicon | |
|---|---|---|---|---|---|---|---|---|
| SEQ | Marker | | | distance | Insert | | Size | |
| NUM | Name | Primer Name | Chromosome | (cM) | Col | Ler | Col | Ler |
| 1483 | 460499 | 460499_F | 2 | 85 | 19 | 0 | 216 | 197 |
| 1484 | 460499 | 460499_R | 2 | 85 | 19 | 0 | 216 | 197 |
| 1485 | 456794 | 456794_F | 5 | 96 | 14 | 0 | 182 | 168 |
| 1486 | 456794 | 456794_R | 5 | 96 | 14 | 0 | 182 | 168 |
| 1487 | nga6 | nga6_F | 3 | 85 | 19 | 0 | 161 | 142 |
| 1488 | nga6 | nga6_R | 3 | 85 | 19 | 0 | 161 | 142 |
| 1489 | nga63 | nga63_F | 1 | 9 | 25 | 0 | 129 | 104 |
| 1490 | nga63 | nga63_R | 1 | 9 | 25 | 0 | 129 | 104 |
| 1491 | 461064 | 461064_F | 1 | 2 | 47 | 0 | 377 | 330 |
| 1492 | 461064 | 461064_R | 1 | 2 | 47 | 0 | 377 | 330 |
| 1493 | 457984 | 457984_F | 4 | 16 | 12 | 0 | 238 | 226 |
| 1494 | 457984 | 457984_R | 4 | 16 | 12 | 0 | 238 | 226 |
| 1495 | 452585 | 452585_F | 1 | 90 | 56 | 0 | 396 | 340 |
| 1496 | 452585 | 452585_R | 1 | 90 | 56 | 0 | 396 | 340 |
| 1497 | 454039 | 454039_F | 5 | 51 | 35 | 0 | 199 | 164 |
| 1498 | 454039 | 454039_R | 5 | 51 | 35 | 0 | 199 | 164 |
| 1499 | 455075 | 455075_F | 5 | 23 | 0 | 15 | 143 | 158 |
| 1500 | 455075 | 455075_R | 5 | 23 | 0 | 15 | 143 | 158 |
| 1501 | nga280 | nga280_F | 1 | 81 | 18 | 0 | 122 | 104 |
| 1502 | nga280 | nga280_R | 1 | 81 | 18 | 0 | 122 | 104 |
| 1503 | AthCDC2BGR | AthCDC2BGR_F | 3 | 74 | 2 | 0 | 147 | 145 |
| 1504 | AthCDC2BGR | AthCDC2BGR_R | 3 | 74 | 2 | 0 | 147 | 145 |
| 1505 | 455469 | 455469_F | 3 | 43 | 18 | 0 | 207 | 189 |
| 1506 | 455469 | 455469_R | 3 | 43 | 18 | 0 | 207 | 189 |
| 1507 | 466785 | 466785_F | 5 | 110 | 24 | 0 | 267 | 243 |
| 1508 | 466785 | 466785_R | 5 | 110 | 24 | 0 | 267 | 243 |
| 1509 | nga162 | nga162_F | 3 | 21 | 17 | 0 | 123 | 106 |
| 1510 | nga162 | nga162_R | 3 | 21 | 17 | 0 | 123 | 106 |
| 1511 | nga172 | nga172_F | 3 | 6 | 25 | 0 | 179 | 154 |
| 1512 | nga172 | nga172_R | 3 | 6 | 25 | 0 | 179 | 154 |
| 1513 | 466780 | 466780_F | 2 | 19 | 55 | 0 | 452 | 397 |
| 1514 | 466780 | 466780_R | 2 | 19 | 55 | 0 | 452 | 397 |
| 1515 | 450584 | 450584_F | 1 | 108 | 12 | 0 | 116 | 104 |
| 1516 | 450584 | 450584_R | 1 | 108 | 12 | 0 | 116 | 104 |
| 1517 | 453494 | 453494_F | 1 | 115 | 0 | 16 | 155 | 171 |
| 1518 | 453494 | 453494_R | 1 | 115 | 0 | 16 | 155 | 171 |
| 1519 | 466786 | 466786_F | 5 | 130 | 0 | 14 | 130 | 144 |
| 1520 | 466786 | 466786_R | 5 | 130 | 0 | 14 | 130 | 144 |
| 1521 | 449044 | 449044_F | 4 | 72 | 37 | 0 | 214 | 177 |
| 1522 | 449044 | 449044_R | 4 | 72 | 37 | 0 | 214 | 177 |
| 1523 | 452443 | 452443_F | 1 | 99 | 15 | 0 | 279 | 264 |
| 1524 | 452443 | 452443_R | 1 | 99 | 15 | 0 | 279 | 264 |
| 1525 | 455725 | 455725_F | 5 | 120 | 25 | 0 | 373 | 348 |
| 1526 | 455725 | 455725_R | 5 | 120 | 25 | 0 | 373 | 348 |
| 1527 | 455913 | 455913_F | 3 | 29 | 3 | 0 | 116 | 113 |
| 1528 | 455913 | 455913_R | 3 | 29 | 3 | 0 | 116 | 113 |
| 1529 | nga168 | nga168_F | 2 | 73 | 16 | 0 | 169 | 153 |
| 1530 | nga168 | nga168_R | 2 | 73 | 16 | 0 | 169 | 153 |
| 1531 | nga8 | nga8_F | 4 | 24 | 0 | 43 | 174 | 217 |
| 1532 | nga8 | nga8_R | 4 | 24 | 0 | 43 | 174 | 217 |
| 1533 | 459322 | 459322_F | 2 | 49 | 0 | 43 | 427 | 470 |
| 1534 | 459322 | 459322_R | 2 | 49 | 0 | 43 | 427 | 470 |
| 1535 | 453973 | 453973_F | 4 | 54 | 27 | 0 | 407 | 380 |
| 1536 | 453973 | 453973_R | 4 | 54 | 27 | 0 | 407 | 380 |
| 1537 | 466787 | 466787_F | 5 | 88 | 21 | 0 | 369 | 348 |
| 1538 | 466787 | 466787_R | 5 | 88 | 21 | 0 | 369 | 348 |
| 1539 | nga225 | nga225_F | 5 | 11 | 0 | 72 | 136 | 208 |
| 1540 | nga225 | nga225_R | 5 | 11 | 0 | 72 | 136 | 208 |
| 1541 | nga76 | nga76_F | 5 | 71 | 0 | 69 | 244 | 313 |
| 1542 | nga76 | nga76_R | 5 | 71 | 0 | 69 | 244 | 313 |
| 1543 | 466776 | 466776_F | 1 | 48 | 0 | 27 | 399 | 426 |
| 1544 | 466776 | 466776_R | 1 | 48 | 0 | 27 | 399 | 426 |
| 1545 | 453574 | 453574_F | 4 | 48 | 0 | 30 | 420 | 450 |
| 1546 | 453574 | 453574_R | 4 | 48 | 0 | 30 | 420 | 450 |
| 1547 | 455553 | 455553_F | 5 | 7 | 0 | 32 | 211 | 243 |
| 1548 | 455553 | 455553_R | 5 | 7 | 0 | 32 | 211 | 243 |
| 1549 | 456131 | 456131_F | 5 | 79 | 0 | 32 | 283 | 315 |
| 1550 | 456131 | 456131_R | 5 | 79 | 0 | 32 | 283 | 315 |
| 1551 | 457489 | 457489_F | 5 | 42 | 0 | 28 | 156 | 184 |
| 1552 | 457489 | 457489_R | 5 | 42 | 0 | 28 | 156 | 184 |
| 1553 | 460464 | 460464_F | 3 | 62 | 0 | 18 | 321 | 339 |
| 1554 | 460464 | 460464_R | 3 | 62 | 0 | 18 | 321 | 339 |
| 1555 | 450660 | 450660_F | 5 | 60 | 16 | 0 | 218 | 202 |
| 1556 | 450660 | 450660_R | 5 | 60 | 16 | 0 | 218 | 202 |
| 1557 | 458709 | 458709_F | 2 | 10 | 42 | 0 | 452 | 410 |
| 1558 | 458709 | 458709_R | 2 | 10 | 42 | 0 | 452 | 410 |
| 1559 | AthSO392 | AthSO392_F | 1 | 40 | 0 | 16 | 158 | 174 |
| 1560 | AthSO392 | AthSO392_R | 1 | 40 | 0 | 16 | 158 | 174 |
| 1561 | 466777 | 466777_F | 3 | 51 | 4 | 0 | 189 | 185 |
| 1562 | 466777 | 466777_R | 3 | 51 | 4 | 0 | 189 | 185 |
| 1563 | 459775 | 459775_F | 2 | 63 | 68 | 0 | 426 | 358 |
| 1564 | 459775 | 459775_R | 2 | 63 | 68 | 0 | 426 | 358 |
| 1565 | 466781 | 466781_F | 2 | 40 | 0 | 7 | 213 | 220 |
| 1566 | 466781 | 466781_R | 2 | 40 | 0 | 7 | 213 | 220 |
| 1567 | 466778 | 466778_F | 2 | 2 | 19 | 0 | 283 | 264 |
| 1568 | 466778 | 466778_R | 2 | 2 | 19 | 0 | 283 | 264 |
| 1569 | CZS0D2 | CZS0D2_F | 2 | 56 | 20 | 0 | 204 | 184 |
| 1570 | CZS0D2 | CZS0D2_R | 2 | 56 | 20 | 0 | 204 | 184 |
| 1571 | nga106 | nga106_F | 5 | 33 | 33 | 0 | 171 | 138 |
| 1572 | nga106 | nga106_R | 5 | 33 | 33 | 0 | 171 | 138 |
| 1573 | 450129 | 450129_F | 1 | 32 | 27 | 0 | 392 | 365 |
| 1574 | 450129 | 450129_R | 1 | 32 | 27 | 0 | 392 | 365 |
| 1575 | 466788 | 466788_F | 4 | 7 | 39 | 0 | 271 | 232 |
| 1576 | 466788 | 466788_R | 4 | 7 | 39 | 0 | 271 | 232 |
| 1577 | 454879 | 454879_F | 5 | 132 | 12 | 0 | 299 | 287 |
| 1578 | 454879 | 454879_R | 5 | 132 | 12 | 0 | 299 | 287 |
| 1579 | nga1107 | nga1107_F | 4 | 102 | 20 | 0 | 168 | 148 |
| 1580 | nga1107 | nga1107_R | 4 | 102 | 20 | 0 | 168 | 148 |
| 1581 | 466779 | 466779_F | 4 | 5 | 0 | 49 | 306 | 355 |
| 1582 | 466779 | 466779_R | 4 | 5 | 0 | 49 | 306 | 355 |

### EXAMPLE 4

PCR primers can be designed for the flanking sequence of polymorphisms and can be used to either confirm or detect the polymorphisms. Such primers are designed with the program Primer3 (obtained from the MIT-Whitehead Genome Center) with a “perl-oracle” wrapper. The criteria applied to design a primer include:

Primer annealing temperature (minimum 57° C., optimum 60° C., maximum 63° C.)

Primer length (minimum 18 bp, optimum 20 bp, maximum 27 bp)

G+C content (minimum 20%, maximum 80%)

Minimum target margin of the primer relative to the polymorphism: 50 bp

Length of the amplified region
for SNPs: minimum 480 bp, optimum 500 bp, maximum 550 bp
for INDELs: minimum 200 bp, optimum 400 bp, maximum 500 bp

PHRED quality score of the gene template (minimum of 0)

Target sequence on one contig

Maximum mismatch=12.0 (weighted score from Primer3 program)

Pair Max Misprime=24.0 (weighted score from Primer3 program)

Maximum N's=0

Maximum poly-X=5

The primary goal of the design process is the creation of groups of primer pairs with a common annealing temperature (Tₘ).

After the Arabidopsis thaliana specific portion of the primers is selected, an additional common primer tail sequence can be added to the 5′ ends. Forward primers for the detection of insertion/deletion polymorphisms have the additional common M13 bases on the 5′ end: (5′-CAGCACGTTGTAAAACGAC-3′); reverse primers for the detection of insertion/deletion polymorphisms were designed without a tail. Forward primers for the detection of SNPs have the additional common M13 bases on the 5′ end: (5′-TGTAAAACGACGGCCAGTT-3′); reverse primers for SNPs have the additional common M13 bases on the 5′ end: (5′-CAGGAAACAGCTATGACC-3′). The primer tail sequences are added so that subsequent amplifications of any primer pair can be done with a specific kit designed to work with oligonucleotides having the primer tail. It is noted that primer pairs are not required to contain the tail sequence, the relevant portion for amplification and/or hybridization probes being the Arabidopsis thaliana specific sequences.

Using such primers for polymorphic marker flanking sequence, a person skilled in the art can amplify genetic regions from Arabidopsis thaliana, Columbia and Arabidopsis thaliana, Landsberg erecta genomic DNA, as well as from a mixture of Arabidopsis thaliana, Columbia and Arabidopsis thaliana, Landsberg erecta genomic DNA to represent a heterozygote. In the case of SNPs the amplified product is purified and sequenced to confirm the presence of a predicted SNP. For validation of INDELs, the amplified products are analyzed or sized on an agarose gel or an acrylamide gel to determine if the fragments amplified from Arabidopsis thaliana, Columbia and Arabidopsis thaliana, Landsberg erecta genomic DNA are polymorphic. An exemplary PCR amplification reaction procedure to detect an INDEL-type polymorphism in a mapping experiment is a follows: a reaction mixture containing 4 ng/μl DNA (2.6 μl); Taq Gold Polymerase (5 units/μl) (0.1 μl) (Perkin Elmer, Norwalk, Conn.); 5 μm forward and reverse primer (0.2 μl); 1 ηm Li-Cor M13 Forward/IRD 700 (0.5 μl)(Lincoln, Neb.); 50 mM MgCl₂ (0.3 μl); 10 mM dNTPs (2.5 mM each of dCTP, dGTP, dATP and dTTP)(0.8 μl); 10× Taq Gold Buffer (1.0 μl); dH₂0 (4.5 μl). Thermal amplification is carried out in an MJ Tetrad as follows: 94° C. 10 minutes; 35 cycles (94° C. 1 minute, 56° C. 1 minute, 72° C. 1 minute); 72° C. 10 minutes; 4° C. hold. PCR products are loaded on a 7% Long Ranger gel and run on Li-Cor's DNA Sequencer Long Redir 4200 or DNA Analyzer Gene Readir 4200 according to manufacturer's protocol. Data is analyzed using GenelmagIR software.

An exemplary PCR amplification reaction to detect a SNP-type polymorphism in a mapping experiment is a follows: A reaction mixture containing 4 ng/μl DNA (6.6 μl); 5 units Platinum Gold Polymerase (5 units/μl)(0.1 μl) (GibcoBRL, Rockville, Md. (0.1 μl); 5 μm forward and reverse primer with M13 tails (1.39 μl); 50 mM MgCl₂ (0.66 μl); 10 mM dNTPs (2.5 mM each of dCTP, dGTP, dATP and dTTP)(1.04 μl); 10× Taq Platinum Buffer (2.43 μl); dH₂O (12.77 μl). Thermal amplification is carried out in an MJ Tetrad as follows: 94° C. 10 minutes; 35 cycles (94° C. 1 minute, 56° C. 1 minute, 72° C. 1 minute); 72° C. 10 minutes; 4° C. hold. PCR products are purified using QIAGEN's QIAquick 96 PCR Purification Kit as per manufactures' protocol. Purified PCR products are run on agarose gels to confirm amplification, followed by sequencing to confirm the presences of a SNP.

1. A method of isolating a nucleic acid molecule associated with a phenotype trait comprising:
(A) screening a mapping population of Arabidopsis plants to determine the linkage of said phenotypic trait with a collection of polymorphisms, wherein at least one polymorphism is selected from Table A; and
(B) isolating said nucleic acid molecule associated with said phenotypic trait based on its linkage to one or more of said polymorphisms.

2. A collection of non-identical nucleic acid molecules capable of detecting polymorphisms present in an Arabidopsis mapping population, wherein said collection of non-identical nucleic acid molecules is capable of detecting at least 25 polymorphisms selected from the group consisting of Table A, and wherein each non-identical nucleic acid molecule in the collection comprises at least 12 nucleotides.

3. The collection of non-identical nucleic acid molecules according to claim 2, wherein said collection is fabricated onto a substrate.

4. A method of identifying a nucleic acid molecule associated with a phenotypic trait of interest comprising:
(A) screening a mapping population of Arabidopsis plants to determine the linkage of said phenotypic trait with a collection of polymorphisms, wherein said polymorphisms are distributed throughout the genome of said mapping population of Arabidopsis plants wherein at least one of the polymorphisms is selected from Table A;
(B) calculating the linkage of each said polymorphism to said phenotypic trait; and,
(C) isolating said nucleic acid molecule associated with said phenotypic trait based on its linkage to one or more of said polymorphisms,
and wherein said collection of polymorphisms is designed from features of polymorphisms listed in Table A and recorded on computer readable medium.

5. The collection of non-identical nucleic acid molecules according to claim 2, wherein said collection is fabricated on a substrate and the location of each nucleic acid molecule in the collection is recorded on a computer readable medium.

6. The collection of non-identical nucleic acid molecules of claim 3, wherein said substrate is embedded in a microarray, and wherein said polymorphisms comprise at least twenty-five polymorphisms selected from the group consisting of polymorphism Nos: 470913, 469906, 470827, 470828, 470268, 470145, 470959, 470908, 469866, 469869, 469872, 469874, 470478, 470529, 470920, 470409, 470499, 469600, 469859, 470754, 469498, 470033, 470283, 473818, and 469665.

7. The collection of non-identical nucleic acid molecules of claim 5, wherein said substrate is embedded in a microarray, wherein said polymorphisms comprise at least twenty-five polymorphisms selected from the group consisting of polymorphism Nos: 470913, 469906, 470827, 470828, 470268, 470145, 470959, 470908, 469866, 469869, 469872, 469874, 470478, 470529, 470920, 470409, 470499, 469600, 469859, 470754, 469498, 470033, 470283, 473818, and 469665.

8. The collection of non-identical nucleic acid molecules according to claim 2, wherein said polymorphisms include polymorphism No: 470268.

9. The collection of non-identical nucleic acid molecules according to claim 2, wherein said polymorphisms include polymorphism No: 469600.
